(19) United States
(12) Patent Application Publication
(10) Pub. No.: US 2021/0307845 A1
(43) Pub. Date: Oct. 7, 2021

(54) GENERATION OF GRAPHICAL REPRESENTATION OF FORCE

(71) Applicant: Intuitive Surgical Operations, Inc., Sunnyvale, CA (US)

(72) Inventors: June Gyu Park, San Jose, CA (US); Matthew D. Rohr Daniel, San Francisco, CA (US); Nicola Diolaiti, Menlo Park, CA (US); Troy K. Adebar, Mountain View, CA (US); Cristian Bianchi, Los Gatos, CA (US)

(21) Appl. No.: 17/274,735

(22) PCT Filed: Sep. 11, 2019
(86) PCT No.: PCT/US2019/050571

§ 371 Date: Mar. 9, 2021

Related U.S. Application Data
(60) Provisional application No. 62/729,877, filed on Sep. 11, 2018.

(51) Int. Cl. A61B 34/00; A61B 90/00; A61B 17/00; A61B 34/20

(52) U.S. Cl. CPC A61B 34/25; A61B 90/06; A61B 2034/301; A61B 34/20; A61B 17/00234

(57) ABSTRACT

A machine is configured to access force data generated by a force sensor, where the force data quantifies variation in magnitude of a force encountered by a distal portion of an elongate device during a time period in which the distal portion traveled a distance within an environment during a procedure, and where the force data includes a current value of the magnitude of the force. Based on the force data, the machine generates a graphical representation that indicates the force encountered by the distal portion at a point in the procedure relative to an entirety of the procedure, and then causes a display screen to present the graphical representation. Other apparatus and methods are also described.

### RELATED APPLICATION

This application claims the priority benefit of U.S. Provisional Patent Application No. 62/729,877, filed Sep. 11, 2018, which is incorporated herein by reference in its entirety,

### TECHNICAL FIELD

The subject matter disclosed herein generally relates to the technical field of special-purpose machines that facilitate human control of a device (e.g., a robot or other device), including software-configured computerized variants of such special-purpose machines and improvements to such variants, and to the technologies by which such special-purpose machines become improved compared to other special-purpose machines that facilitate human control of devices. Specifically, the present disclosure addresses systems and methods that, in the process of facilitating human control of a device, facilitate generation of a graphical representation of a force encountered by the device.

### BACKGROUND

Minimally invasive medical techniques are intended to reduce the amount of tissue that is damaged during medical procedures, thereby reducing patient recovery time, discomfort, and harmful side effects. Such minimally invasive techniques may be performed through natural orifices in a patient's anatomy or through one or more surgical incisions. Through these natural orifices or incisions, surgeons or other clinicians may insert minimally invasive medical instruments (e.g., surgical, diagnostic, therapeutic, or biopsy instruments) to reach a target tissue location. One such minimally invasive technique is to use a flexible, steerable elongate device e.g., a catheter) that can be inserted into anatomic passageways and navigated toward a region of interest within the patient's anatomy. Control of such an elongate device by medical personnel during an image-guided procedure involves the management of several degrees of freedom, which may include managing insertion or retraction of the elongate device, steering the device, managing the bend radius of the device, or any suitable combination thereof.

Accordingly, it would be beneficial to provide a graphical user interface (GUI) that supports intuitive control and management of medical instruments, including elongate devices, such as flexible and steerable catheters, that are suitable for performing minimally invasive medical techniques.

### SUMMARY

Example embodiments discussed herein are best summarized by the claims that follow the description.

In some example embodiments, a system includes: a flexible elongate device including a distal portion configured to travel within an environment during and a proximal portion configured to remain external to the environment; a force sensor coupled to the proximal portion of the flexible elongate device and configured to generate force data during a procedure; a control system communicatively coupled to the flexible elongate device and the force sensor, the control system being configured to, based on the force data, generate a graphical representation that indicates a force encountered by the distal portion of the flexible elongate device at a point in the procedure relative to an entirety of the procedure; and a display screen communicatively coupled to the control system and configured to present the graphical representation indicative of the force encountered by the distal portion at the point relative to the entirety of the procedure.

In certain example embodiments, a method includes: generating, by one or more processors, force data based on input from a force sensor communicatively coupled to a proximal portion of a flexible elongate device during a procedure in which a distal portion of the flexible elongate device travels within an environment; generating, based on the force data and by one or more of the processors, a graphical representation that indicates a force encountered by a distal portion of the flexible elongate device at a point in the procedure relative to an entirety of the procedure; and causing, by one or more of the processors, a display screen to present the graphical representation indicative of the force encountered by the distal portion at the point relative to the entirety of the procedure.

In various example embodiments, a machine-readable medium includes instructions that, when executed by one or more processors of a machine, cause the machine to perform operations including: generating force data based on input from a force sensor communicatively coupled to a proximal portion of a flexible elongate device during a procedure in which a distal portion of the flexible elongate device travels within an environment; generating, based on the three data, a graphical representation that indicates a force encountered by a distal portion of the flexible elongate device at a point in the procedure relative to an entirety of the procedure; and causing a display screen to present the graphical representation indicative of the force encountered by the distal portion at the point relative to the entirety of the procedure.

In some example embodiments, a system includes: an elongate device including a distal portion configured to travel within an environment; a force sensor coupled to the elongate device and configured to measure a force encountered by the distal portion of the elongate device during a time period in which the distal portion travels a distance within the environment, the force sensor being configured to generate force data based on the measured force; a display screen; one or more processors; and a memory storing instructions that, when executed by at least one processor among the one or more processors, cause the at least one processor to perform operations including: accessing the force data generated by the force sensor, the force data quantifying variation in magnitude of the force encountered by the distal portion of the elongate device during the time period in which the distal portion traveled the distance within the environment, the force data including a current value of the magnitude of the force; determining a temporal rate of change of the force during the time period based on the force data and the time period; determining a spatial rate of change of the force based on the force data and the distance traveled by the distal portion of the elongate device; generating a graphical representation of the force based on the current value of the magnitude of the force, the temporal rate of change of the force, and the spatial rate of change of the force; and causing the display screen to present the graphical representation generated based on the current value, the temporal rate of change, and the spatial rate of change.

In certain example embodiments, a method includes: accessing force data generated by a force sensor, the force data quantifying variation in magnitude of a force encountered by a distal portion of an elongate device during a time period in which the distal portion traveled a distance within an environment, the force data including a current value of the magnitude of the force; determining a temporal rate of change of the force during the time period based on the force data and the time period; determining a spatial rate of change of the force based on the force data and the distance traveled by the distal portion of the elongate device; generating a graphical representation of the force based on the current value of the magnitude of the force, the temporal rate of change of the force, and the spatial rate of change of the force; and causing the display screen to present the graphical representation generated based on the current value, the temporal rate of change, and the spatial rate of change.

In various example embodiments, a machine-readable medium includes instructions that, when executed by one or more processors of a machine, cause the machine to perform operations including: accessing force data generated by a force sensor, the force data quantifying variation in magnitude of a force encountered by a distal portion of an elongate device during a time period in which the distal portion traveled a distance within an environment, the force data including a current value of the magnitude of the force; determining a temporal rate of change of the force during the time period based on the force data and the time period; determining a spatial rate of change of the force based on the force data and the distance traveled by the distal portion of the elongate device; generating a graphical representation of the force based on the current value of the magnitude of the force, the temporal rate of change of the force, and the spatial rate of change of the force; and causing the display screen to present the graphical representation generated based on the current value, the temporal rate of change, and the spatial rate of change.

### BRIEF DESCRIPTION OF THE DRAWINGS

Some embodiments are illustrated by way of example and not limitation in the figures of the accompanying drawings.

FIG. 1 is a diagram illustrating an elongate device being controlled by a control system and its operator (e.g., a surgeon), according to some example embodiments.

FIG. 2 is a diagram illustrating the elongate device and its control system, according to some example embodiments.

FIGS. 3 and 4 are screenshots of a portion of a display screen that is presenting a graphical representation of force, according to some example embodiments.

FIG. 5 is a block diagram illustrating components of the control system for the elongate device, according to some example embodiments.

FIGS. 6-9 are flowcharts illustrating operations of the control system in performing a method of generating a graphical representation of force, according to some example embodiments.

FIGS. 10-12 are flowcharts illustrating operation of the control system in performing another method of generating a graphical representation of force, according to some example embodiments.

FIG. 13 is a block diagram illustrating components of a machine, according to some example embodiments, able to read instructions from a machine-readable medium and perform any one or more of the methodologies discussed herein.

### DETAILED DESCRIPTION

Example methods (e.g., algorithms) facilitate generating a graphical representation of force (e.g., for presentation in a GUI), and example systems (e.g., special-purpose machines configured by special-purpose software) are configured to facilitate generating a graphical representation of force. Examples merely typify possible variations. Unless explicitly stated otherwise, structures (e.g., structural components, such as modules) are optional and may be combined or subdivided, and operations (e.g., in a procedure, algorithm, or other function) may vary in sequence or be combined or subdivided. In the following description, for purposes of explanation, numerous specific details are set forth to provide a thorough understanding of various example embodiments. It will he evident to one skilled in the art, however, that the present subject matter may be practiced without these specific details.

A machine may form all or part of a control system that is configured (e.g., by suitable hardware, software, or both) to interact with one or more users by generating and providing graphical representations of various data (e.g., in real time), including generating and providing a graphical representation of a force. In the example context of a surgeon performing robotic surgery, the machine may be configured to access force data generated by a force sensor, where the force data quantifies variation in magnitude of a force encountered by a distal portion of an elongate device (e.g., a robotic surgical catheter inserted into a human patient) during a time period in which the distal portion traveled a distance within an environment (e.g., the human patient), and where the force data includes a current value of the magnitude of the force. The machine may then determine (e.g., by calculation) two rates of change of the force, namely, a temporal rate of change of the force during the time period and a spatial rate of change of the force over the distance traveled. The temporal rate of change may be determined based on the force data and the time period, and the spatial rate of change may be determined based on the force data and the distance traveled by the distal portion of the elongate device. The machine then generates a graphical representation of the force based on the current value of the magnitude of the force, the temporal rate of change of the force, and the spatial rate of change of the force. The machine may accordingly cause a display screen to present the graphical representation to a user (e.g., the surgeon).

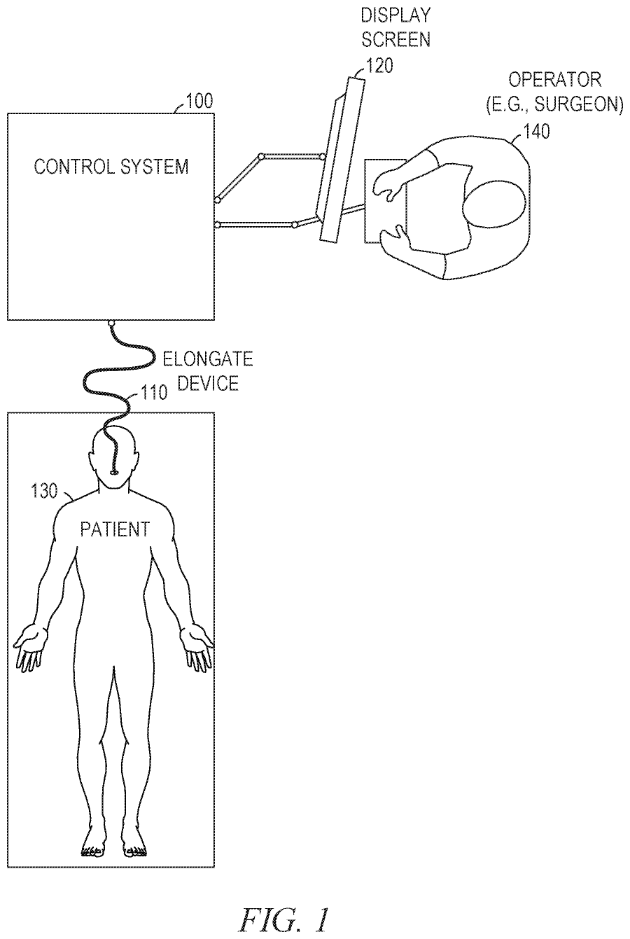

FIG. 1 is a diagram illustrating an elongate device 110 being controlled (e.g., navigated, moved, inserted, withdrawn, or otherwise manipulated) by a control system 100 and its operator 140 (e.g., a surgeon, clinician, or physician), according to some example embodiments. The control system 100 may be or include a teleoperated medical system that is configured or otherwise suitable for use in medical procedures, such as surgical, diagnostic, therapeutic, or biopsy procedures. As shown in FIG. 1, the control system 100 includes and controls (e.g., guides) the elongate device 110, which may form all or part of a medical instrument suitable for performing various medical procedures on a patient 130, in some example embodiments, the proximal portion (e.g., the proximal end or proximate end) of the elongate device 110 is mounted on or near an operating table upon which the patient 130 is lying, while the distal portion (e.g., the distal end) of the elongate device 110 is inserted into the anatomy of the patient 130 (e.g., through an incision therein or an orifice thereof).

The operator 140 may be located at a physician's console, which may be located in the same room as the patient 130, such as at the side of a surgical table on which patient 130 located. However, the operator 140 may be located in a different room or a completely different building from the patient 130. The control system 100 may include one or more control devices for controlling the elongate device 110 (e.g., by actuating one or more actuators within the elongate device 110). Such control devices may be or include various input devices, such as joysticks, trackballs, data gloves, trigger-guns, hand-operated controllers, voice recognition devices, body motion or presence sensors, or any suitable combination thereof. To provide the operator 140 with a strong sense of directly controlling the elongate device 110, such control devices may be configured to operate with the same degrees of freedom as the elongate device 110. In this manner, the control devices provide operator 140 with telepresence or the perception that the control devices are integral with the elongate device 110.

In some example embodiments, the control devices may have more or fewer degrees of freedom than the elongate device 110 and still provide the operator 140 with the above-described telepresence. According to certain embodiments, the control devices may optionally be manual input devices which move with six degrees of freedom, and which may also include an actuation handle for actuating one or more instruments at the distal portion of the elongate device 110 (e.g., for closing grasping jaws, applying an electrical potential to an electrode, delivering a medicinal treatment, or any suitable combination thereof).

The control system 100 additionally controls the display screen 120, which may be configured to display an image or other representation of the surgical site and the elongate device 110. Such an image may be generated by the control system 100. The display screen 120 may be oriented so that the operator 140 can control the elongate device 110 with the perception of telepresence.

In certain example embodiments, the elongate device 110 may include a visualization system, such as a viewing scope assembly that records a current (e.g., real-time) image of a surgical site and provides the image to the operator 140 through one or more displays, such as the display screen 120. For example, the current image may be a two-dimensional or three-dimensional image captured by an endoscope positioned (e.g., by the distal portion of the elongate device 110) within the surgical site. According to some embodiments, the visualization system includes endoscopic components that may be integrally or removably coupled to elongate device 110. However, in some embodiments, a separate endoscope, attached to a separate manipulation device, may be used with the elongate device 110 to image the surgical site. The visualization system may be implemented as hardware, firmware, software, or any suitable combination thereof, which interact with or are otherwise executed by one or more computer processors, which may include one or more processors of the control system 100.

The display screen 120 may display an image of the surgical site and medical instruments captured by the visualization system. In addition, the display screen 120 may present one or more images of a surgical site recorded pre-operatively or intra-operatively using image data from imaging technology such as computed tomography (CT), magnetic resonance imaging (MRI), fluoroscopy, thermography, ultrasound, optical coherence tomography (OCT), thermal imaging, impedance imaging, laser imaging, nanotube X-ray imaging, or any suitable combination thereof. The pre-operative or intra-operative image data may be presented as two-dimensional, three-dimensional, or four-dimensional (e.g., showing time-based or velocity-based information) images, as images that depict one or more models of the patient 130 or a portion of the anatomy thereof, or both.

The display screen 120 may display a navigational image in which the actual location of the elongate device 110 (e.g., the distal portion thereof) is shown with pre-operative or current images. This may be done to present the operator 140 with a navigational image of the internal surgical site from the viewpoint of the elongate device 110 (e.g., the distal portion thereof). In some example embodiments, the viewpoint may be the view looking forward from a tip of the elongate device 110 (e.g., the distal end thereof). An image of the tip of the elongate device 110 (e.g., along with one or more other graphical or alphanumeric indicators) may be superimposed on the navigational image to assist the operator 140 in controlling the elongate device 110. In certain example embodiments, the elongate device 110 is not visible in the navigational image. In certain example embodiments, the viewpoint may be the view looking towards the patient 130 from an external location. An image of all or a portion of the elongate device 110 (e.g., along with one or more other graphical or alphanumeric indicators) may be superimposed on the navigational image to assist the operator 140 in controlling the elongate device 110.

The elongate device 110 may extend into an internal surgical site within the anatomy (e.g., the body) of the patient 130 via an opening (e.g., an incision or in orifice) in the anatomy of the patient 130. The control system 100 may receive feedback from the elongate device 110 (e.g., force, torque, shape, position, velocity, or any suitable combination thereof). Responsive to the feedback, the control system 100 may cause the display screen 120 to present one or more graphical representations of the feedback within a GUI.

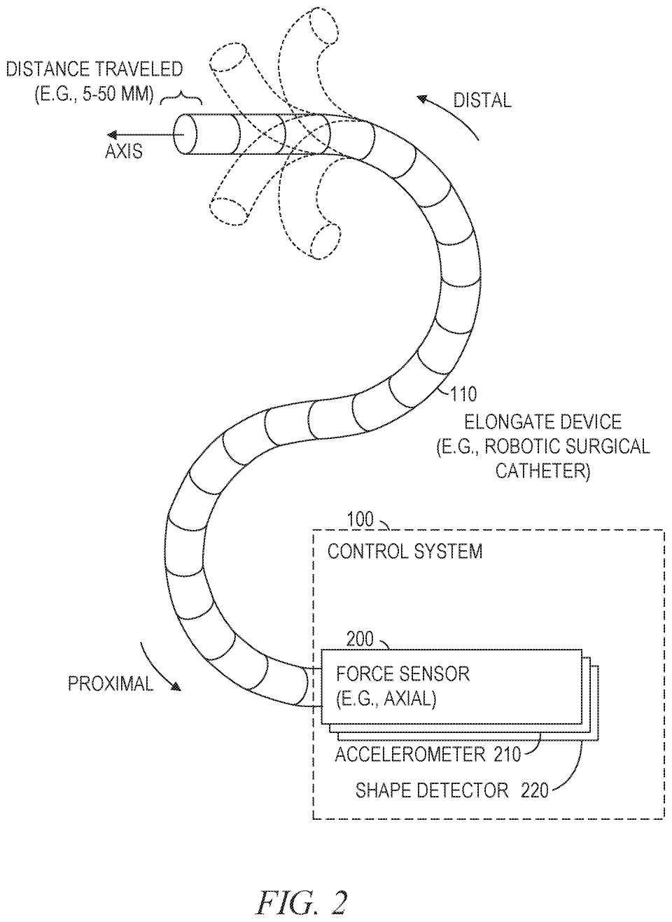

FIG. 2 is a diagram illustrating the elongate device 110 and its control system 100, according to some example embodiments. The elongate device 110 is illustrated in the example form of a robotic surgical catheter and includes a flexible body that has a proximal portion (e.g., terminating in a proximal end) and a distal portion (e.g., terminating in a distal end or distal tip). Accordingly, the elongate device 110 may have an axis that runs the length of the elongate device 110 in an axial direction from the proximal portion to the distal portion. In some example embodiments, the elongate device 110 has an outer diameter of approximately 3 millimeters, though other outer diameters are contemplated. In some example embodiments, the elongate device 110 includes multiple body segments which may be individually or collect actuated to provide flexibility in movement.

As shown in FIG. 2, the distal portion (e.g., the distal tip) of the elongate device 110 can be controllably navigated within an environment (e.g., within the anatomy of the patient 130), such that the distal portion travels an incremental distance (e.g., 5-50 millimeters) through the environment. FIG. 2 fluffier depicts the control system 100 as including a force sensor 200 (e.g., an axial force sensor an accelerometer 210, and a shape detector 220. The force sensor 200 may be coupled (e.g., mechanically, communicatively, or both) to the elongate device 110 and configured to detect (e.g., measure) a force encountered by at least the distal portion of the elongate device 110 (e.g., with or without gravitational and frictional forces acting on the entirety of the elongate device 110). The accelerometer 210 may be coupled to the elongate device 110 and configured to detect (e.g., measure) acceleration (e.g., due to gravity, friction, or both) encountered by the elongate device 110 or a portion thereof. The shape detector 220 may be coupled to the elongate device 110 and configured to detect a shape of the elongate device 110 (e.g., detecting positions, orientations, or both of multiple segments of the elongate device 110). One or both of the accelerometer 210 and the shape detector 220 may be located in any suitable location within or coupled to the elongate device 110, including the example locations shown in FIG. 2.

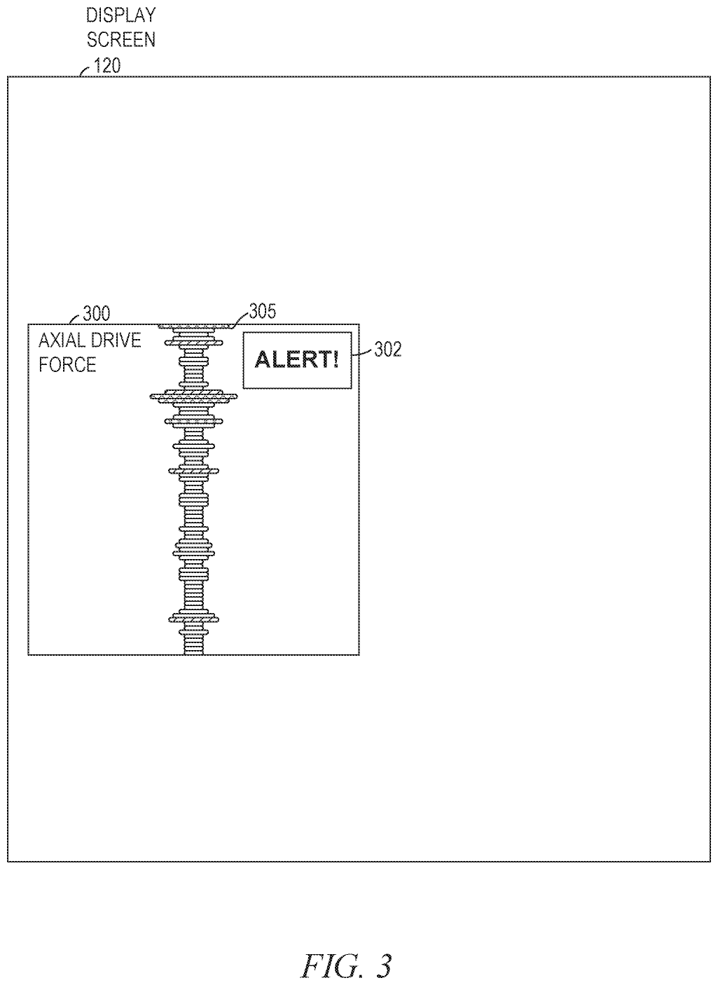

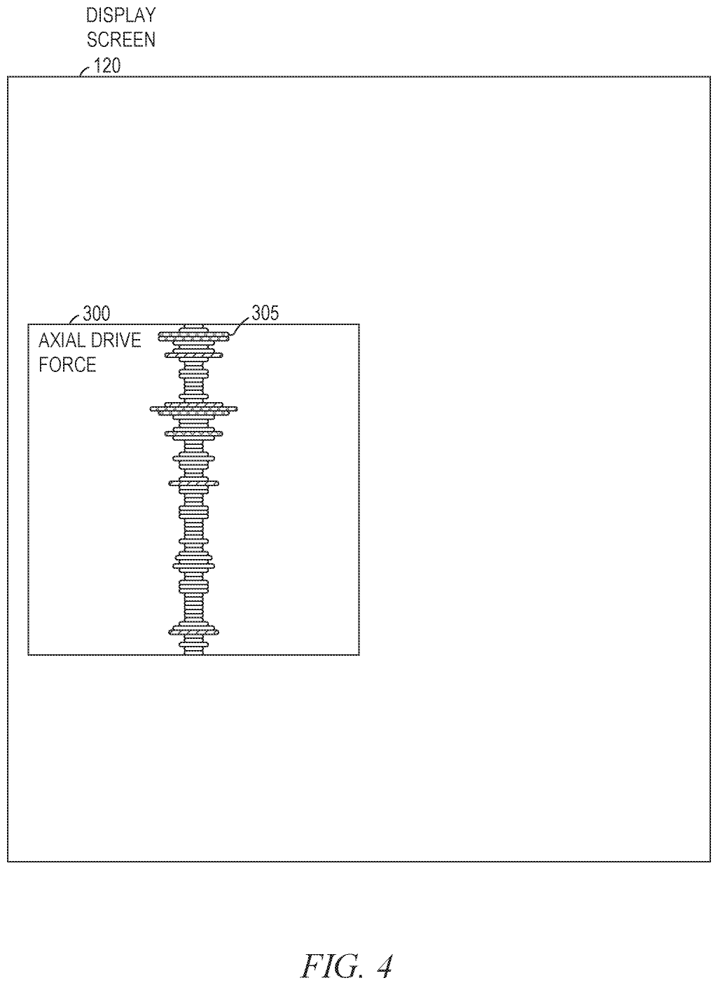

FIGS. 3 and 4 are screenshots of a portion of the display screen 120 that is presenting a graphical representation 300 of force, according to some example embodiments. As shown in FIGS. 3 and 4, the graphical representation 300 takes the example form of a window that forms all or part of a GUI presented by the display screen 120. Such a GUI may provide the operator 140 with some or all of the visual feedback generated by the control system 100 (e.g., in controlling the elongate device 110 through the process of performing a medical procedure on the patient 130). Accordingly, in the example embodiments illustrated in FIGS. 3 and 4, the graphical representation 300 may present the operator 140 with visual feedback that is indicative or otherwise based on a force encountered by the elongate device 110 or a portion thereof (e.g., an axial force encountered by the distal portion of the elongate device 110).

As shown in FIG. 3, at a particular point in time, the graphical representation 300 may present current information about the current value of the magnitude of the force encountered by the elongate device 110, as well as historical information about historical values of the magnitude of the force. For example, the graphical representation 300 may show live information in real-time, as well as a sliding portion of the most recent historical information (e.g., within the past 5-30 seconds). In FIG. 3, the graphical representation 300 includes a bar 305, which as illustrated is the most current (e.g., latest) bar within a sequence of bars that each represent the magnitude of the force during a corresponding period of time (e.g., an incremental period of time in which the distal portion of the elongate device 110 traveled an incremental distance). The bar 305 may form all or part of a portion of the graphical representation 300, and this portion may be colored (e.g., highlighted with a contrasting or other attention-attracting color) based on the current value of the magnitude of the three, the temporal rate of change of the force, the spatial rate of change of the force, or any suitable combination thereof.

Furthermore, although the bar 305 is shown in FIG. 3 as a symmetric indicator of the current value of the magnitude of the force, certain example embodiments utilize an asymmetrical variant of the bar 305. For example, the bar 305 may extend to the right of a marked or unmarked axis to indicate one direction (e.g., insertion) of the force, and the bar 305 may extend to the left of the axis to indicate another direction (e.g., retraction or otherwise opposing) of the force.

According to various example embodiments, the sequence of bars is slowly moving in one axis (e.g., vertically downward) within the graphical representation 300, and the size of each bar (e.g., bar 305) is linearly or non-linearly representative of the current value of the magnitude of the force during the corresponding period of time. For example, the cross-axis (e.g., horizontal) length of the bar 305 may be linearly proportional to the current value of the magnitude of the force, based on the current value falling within a predetermined range of values (e.g., above a minimum threshold value, below a maximum threshold value, or both), and the cross-axis length of the bar 305 may be non-linearly representative (e.g., upscaled or downscaled) of the current value of the magnitude of the force, based on the current value falling outside such a predetermined range of values. in some example embodiments, the axis-aligned (e.g., vertical) length of each bar (e.g., bar 305) is linearly or nonlinearly representative of the duration of the corresponding period of time.

Moreover, in some example embodiments, the cross-axis length of each bar (e.g., bar 305) may be individually adjusted (e.g., when being rendered for addition to the sequence of bars). For example, instead of being scaled based on one or more threshold values, the cross-axis length of a bar (e.g., bar 305) may be determined based on a non-linear scaling function (e.g., ƒ(x)=x³, which attenuates the output when the input is less than unity and amplifies the output when the input exceeds unity).

In addition, FIG. 3 illustrates the graphical representation 300 as including an alert notification 302 (e.g., “ALERT!” or “TISSUE WALL PUNCTURE DANGER!”), which may be presented in response to detection of one or more predetermined conditions. As will be discussed in greater detail below, the control system 100 may detect such conditions based on the current value of the magnitude of the force, the temporal rate of change of the force, the spatial rate of change of the force, or any suitable combination thereof, as well as one or more additional factors (e.g., acceleration data, shape data, or both). Based on such factors, the control system 100 may infer that the distal portion of the elongate device 110 is encountering a blockage, excessive environmental friction, normal environmental friction, or abnormally low environmental friction. Accordingly, presentation of the alert notification may assist the operator 140 in understanding whether the distal portion of the elongate device 110 is being navigated as expected through the environment (e.g., through the anatomy of the patient 130).

As shown in FIG. 4, at a later point in time, the graphical representation 300 may continue to present further current information about the current value of the magnitude of the force encountered by the elongate device 110, as well as historical information about historical values of the magnitude of the force. In the example shown, the graphical representation 300 continues to show live information real-time, as well as the sliding portion of the most recent historical information. Accordingly, the bar 305 is no longer the most current bar within the sequence of bars in the graphical representation 300, though its color may continue to be determined based on the same factors as before (e.g., the then-current value of the magnitude of the force, the then-current temporal rate of change of the force, the then-current spatial rate of change of the force, or any suitable combination thereof). In FIG. 4, the graphical representation 300 no longer shows the alert notification 302. This may be in response to detecting the absence of the one or more predetermined conditions that triggered the presentation of the alert notification shown in FIG. 3.

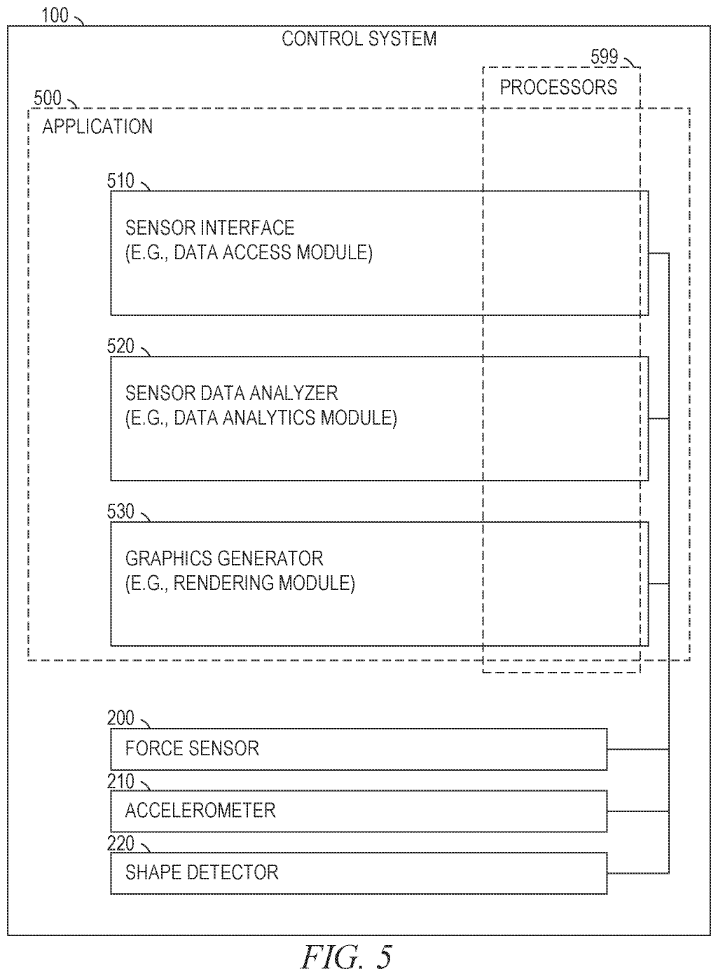

FIG. 5 is a block diagram illustrating components of the control system 100 for the elongate device 110 (e.g., as described above with respect to FIGS. 1 and 2), according to some example embodiments. The control system 100 is shown in the example form of a machine (e.g., a machine that is or includes a computer system) that includes a sensor interface 510, a sensor data analyzer 520, and a graphics generator 530, all configured to communicate with each other (e.g., via a bus, shared memory, or a switch). Additionally, the control system 100 is shown in FIG. 5 as including the force sensor 200, the accelerometer 210, and the shape detector 220, which were introduced above.

The sensor interface 510 may be or include a data access module or similarly suitable code configured to access data from one or more sensors (e.g., the force sensor 200, the accelerometer 210, the shape detector 220, or any suitable combination thereof). The sensor data analyzer 520 may be or include a data analytics module or similarly suitable code configured to analyze the data accessed by the sensor interface 510. The graphics generator 530 may be or include a rendering module (e.g., a GUI rendering module) or similarly suitable code configured to render or otherwise generate graphics, such as the graphical representation 300, which was introduced above.

As shown in FIG. 5, the sensor interface 510, the sensor data analyzer 520, the graphics generator 530, or any suitable combination thereof may form all or part of an application 500 (e.g., a server application, a client application, a mobile app, or any suitable combination thereof) that is stored (e.g., installed) on the control system 100 for execution thereon. Furthermore, one or more processors 599 (e.g., hardware processors, digital processors, or any suitable combination thereof) may be included (e.g., temporarily or permanently) in the application 500, the sensor interface 510, the sensor data analyzer 520, the graphics generator 530, or any suitable combination thereof.

Any one or more of the components (e.g., modules) described herein may be implemented using hardware alone (e.g., one or more of the processors 599) or a combination of hardware and software. For example, any component described herein may physically include an arrangement of one or more of the processors 599 (e.g., a subset of or among the processors 599) configured to perform the operations described herein for that component. As another example, any component described herein may include software, hardware, or both, that configure an arrangement of one or more of the processors 599 to perform the operations described herein for that component. Accordingly, different components described herein may include and configure different arrangements of the processors 599 at different points in time or a single arrangement of the processors 599 at different points in time. Each component (e.g., module) described herein is an example of a means for performing the operations described herein for that component. Moreover, any two or more components described herein may be combined into a single component, and the functions described herein for a single component may he subdivided among multiple components. Furthermore, according to various example embodiments, components described herein as being implemented within a single system or machine (e.g., a single device) may be distributed across multiple systems or machines (e.g., multiple devices).

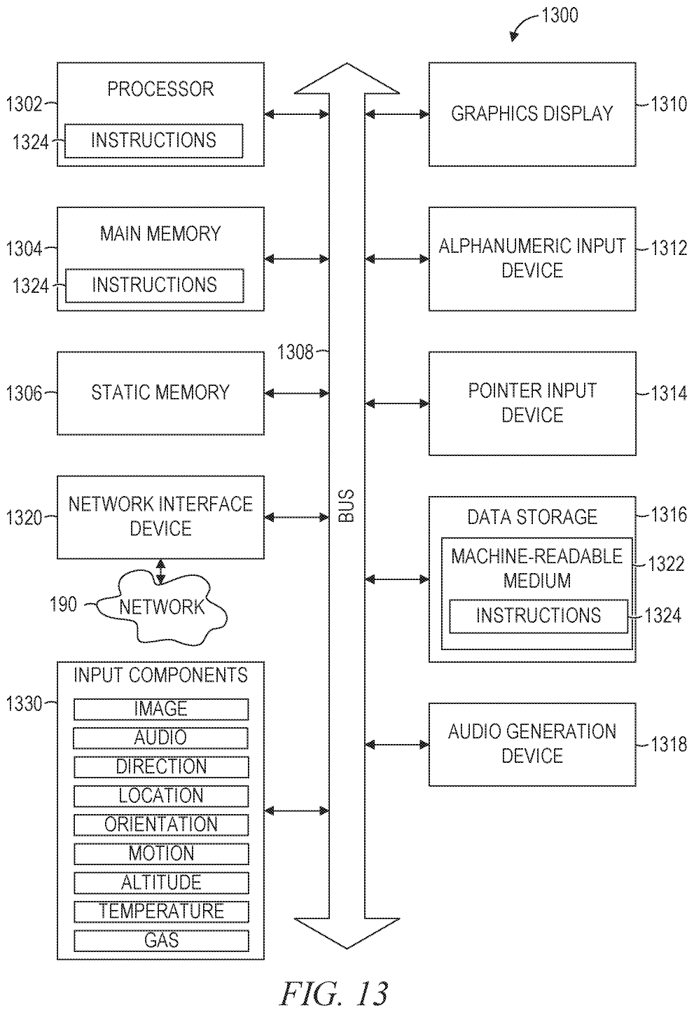

Any of the systems or machines (e.g., devices) discussed herein may be, include, or otherwise be implemented in a special-purpose (e.g., specialized or otherwise non-conventional and non-generic) computer that has been modified to perform one or more of the functions described herein for that system or machine (e.g., configured or programmed by special-purpose software, such as one or more software modules of a special-purpose application, operating system, firmware, middleware, or other software program). For example, a special-purpose computer system able to implement any one or more of the methodologies described herein is discussed below with respect to FIG. 13, and such a special-purpose computer may accordingly he a means for performing any one or more of the methodologies discussed herein. Within the technical field of such special-purpose computers, a special-purpose computer that has been specially modified configured by special-purpose software) by the structures discussed herein to perform the functions discussed herein is technically improved compared to other special-purpose computers that lack the structures discussed herein or are otherwise unable to perform the functions discussed herein. Accordingly, a special-purpose machine configured according to the systems and methods discussed herein provides an improvement to the technology of similar special-purpose machines. Moreover, any two or more of the systems or machines discussed herein may be combined into a single system or machine, and the functions described herein for any single system or machine may be subdivided among multiple systems or machines.

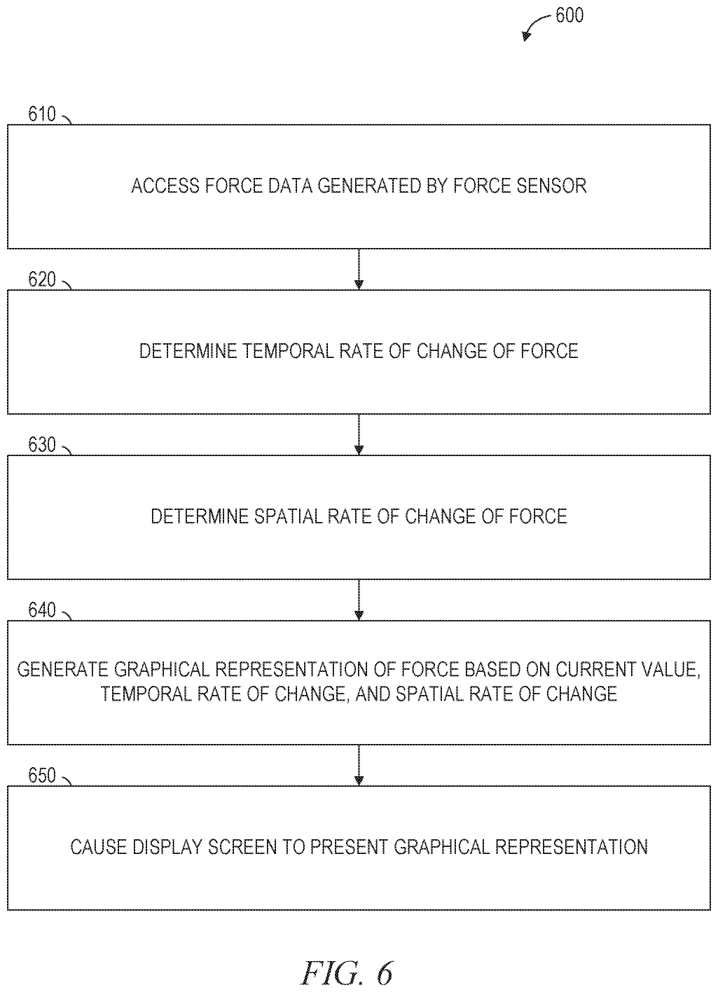

FIGS. 6-9 are flowcharts illustrating operations of the control system 100 (e.g., as described above with respect to FIG. 5, in the context described above with respect to FIGS. 1 and 2, or both) in performing a method 600 of generating the graphical representation 300 of force, according to some example embodiments. Operations in the method 600 may be performed using components (e.g., modules) described above with respect to FIG. 5, using one or more processors (e.g., microprocessors or other hardware processors), or using any suitable combination thereof. As shown in FIG. 6, the method 600 includes operations 610, 620, 630, 640, and 650.

In operation 610, the sensor interface 510 accesses force data generated by the force sensor 200. The force data quantifies variation in magnitude of a force encountered by the distal portion of the elongate device 110. Specifically, the force sensor 200 is configured to measure the force encountered by the distal portion of the elongate device 110, and the force may be encountered and measured during a time period in which the distal portion of the elongate device 110 travels an incremental distance within an environment, such as within the anatomy of the patient 130. The force sensor 200 is further configured to generate the force data based on the measured force. The generated force data accessed in operation 610 may be live (e.g., real-time) three data and accordingly may include a current value of the magnitude of the force.

In operation 620, the sensor data analyzer 520 determines a temporal rate of change of the force during the time period in which the distal portion of the elongate device 110 traveled the incremental distance within the environment. This may be performed by calculating the temporal rate of change of the force based on the force data accessed in operation 610 and the duration of the time period in which the distal portion of the elongate device 110 traveled the incremental distance.

In operation 630, the sensor data analyzer 520 determines a spatial rate of change of the force over the incremental distance traveled by the distal portion of the elongate device 110. This may be performed by calculating the spatial rate of change of the force based on the force data accessed in operation 610 and the traveled incremental distance.

In operation 640, the graphics generator 530 generates the graphical representation 300 of the force encountered by the distal portion of the elongate device 110. This may be performed by generating the graphical representation 300 as all or part of a GUI to be presented by the display screen 120) based on the current value of the magnitude of the force, the temporal rate of change of the force (e.g., as calculated in operation 620), the spatial rate of change of the force (e.g., as calculated in operation 630), or any suitable combination thereof.

In operation 650, the graphics generator 530 causes the display screen 120 to present the graphical representation 300 that was generated in operation 640. This may be performed by providing the generated graphical representation 300 to the display screen 120 (e.g., within a GUI, within a video signal, or both). Accordingly, performance of operation 650 may have the effect of communicating the graphical representation 300 (e.g., with or without an alert notification) to the operator 140 of the control system 100.

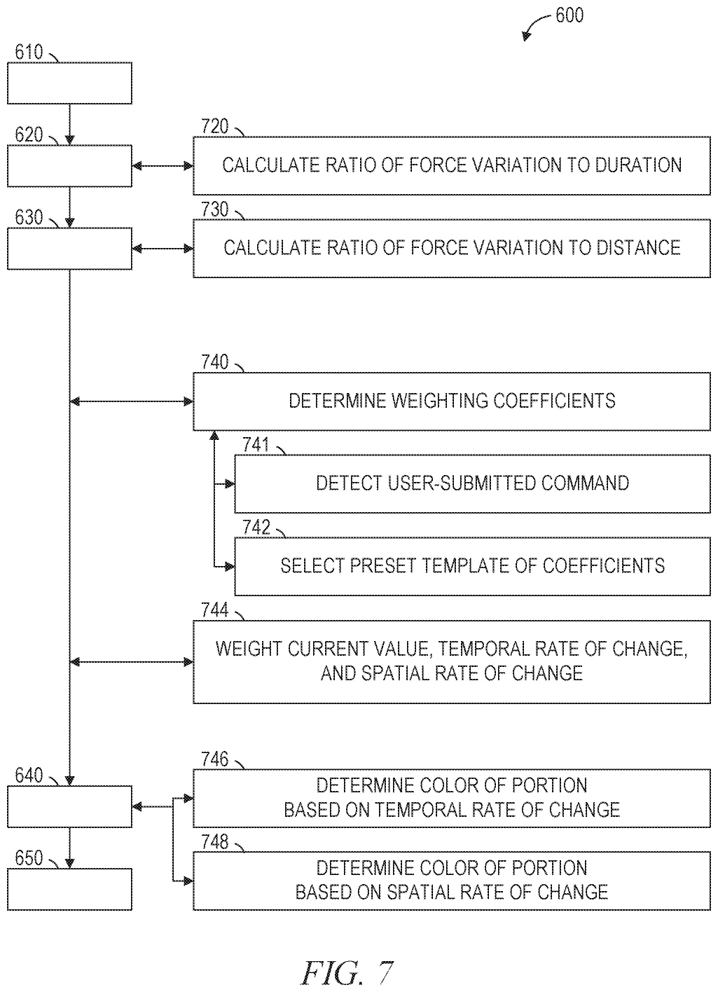

As shown in FIG. 7, in addition to any one or more of the operations previously described, the method 600 may include one or more of operations 720, 730, 740, 741, 742, 744, 746, and 748.

Operation 720 may be performed as part (e.g., a precursor task, a subroutine, or a portion) of operation 620, in which the sensor data analyzer 520 determines the temporal rate of change of the force. In operation 720, the sensor data analyzer 520 calculates a ratio a first ratio or a temporal ratio) of the variation in the magnitude of the force during the time period to the duration of the time period. The variation of the force during the time period in general may be positive or negative. However, in certain example embodiments, the calculation of this ratio uses the absolute value of the variation, while in alternative example embodiments, the positive or negative sign of the variation is preserved through the calculation of the ratio. In example embodiments that include operation 720, the generation of the graphical representation 300 in operation 640 is based on the ratio calculated in operation 720.

Operation 730 may be performed as part of operation 630, in which the sensor data analyzer 520 determines the spatial rate of change of the force. In operation 730, the sensor data analyzer 520 calculates a ratio (e.g., a second ratio or a spatial ratio) of the variation in the magnitude of the force during the time period to the incremental distance traveled by the distal portion of the elongate device 110 during the time period. As noted above, the variation of the force during the time period in general may be positive or negative. However, in certain example embodiments, the calculation of this ratio uses the absolute value of the variation, while in alternative example embodiments, the positive or negative sign of the variation is preserved through the calculation of the ratio. In example embodiments that include operation 730. the generation of the graphical representation 300 in operation 640 is based on the ratio calculated in operation 730.

In some example embodiments, one or more weighting coefficients are applied to the current value of the magnitude of the force, the temporal rate of change in the magnitude of the force e.g., as calculated in operation 620), the spatial rate of change in the magnitude of the force (e.g., as calculated in operation 630), or any suitable combination thereof. In such example embodiments, operations 740 and 744 may be performed at any suitable point prior to operation 640.

In operation 740, the sensor data analyzer 520 determines a set of weighting coefficients to be applied to the current value of the magnitude of the force, the temporal rate of change in the magnitude of the force, and the spatial rate of change in the magnitude of the force.

In certain example embodiments, this set of weighting coefficients is user-specified, and operation 741 may accordingly be performed as part of operation 740. In operation 741, the sensor data analyzer 520 detects a user-submitted command (e.g., submitted by the operator 140) that specifies the set of weighting coefficients to be applied. This detection may be performed by receiving the command or an indication thereof. The specified set of weighting coefficients may correspond to a combination of human health conditions that are specific to the patient 130 (e.g., age, sex, genetics, injury, disease, medical history, diet, exercise, sleep pattern, medication, and the like).

In alternative example embodiments, the set of weighting coefficients is a predetermined set of weighting coefficients, and the predetermined set may be selected from a plurality of multiple predetermined sets of weighting coefficients. For example, the predetermined set may be or include a preset profile that corresponds to a particular situation, such as a particular demographic of the patient 130, a particular health condition to be treated, or a particular preference of the operator 140 for how to operate the elongate device 110. Accordingly, in such alternative example embodiments, operation 742 may be performed as part of operation 740. In operation 742, the sensor data analyzer 520 selects such a predetermined set of weighting coefficients from the plurality of multiple predetermined sets of weighting coefficients. The multiple predetermined sets may each correspond to a different combination of human health conditions, and the selected predetermined set may accordingly correspond to a combination of human health conditions specific to the patient 130.

With the set of weighting coefficients having been determined, in operation 744, the sensor data analyzer 520 mathematically weights the current value of the magnitude of the force, the temporal rate of change in the magnitude of the force, and the spatial rate of change in the magnitude of the force in accordance with their respective weighting coefficients specified in the determined set of weighting coefficients. In example embodiments that include operations 740 and 744, the generating of the graphical representation 300 in operation 640 is based on the weighted current value of the magnitude of the force, the weighted temporal rate of change, and the weighted spatial rate of change.

As shown in FIG. 7, one or both of operations 746 and 748 may be performed as part of operation 640, in which the graphics generator 530 generates the graphical representation 300 of the force. In operation 746, the graphics generator 530 determines a color of at least a portion of the graphical representation 300 (e.g., a group of pixels or a region of the graphical representation 300, such as the alert notification illustrated in FIG. 3), and the color of this portion may be determined based on the temporal rate of change in the magnitude of the force (e.g., as determined in operation 620). Accordingly, in example embodiments that include operation 746, the causing of the display screen 120 to present the graphical representation 300 in operation 650 causes the display screen 120 to present the portion whose color is determined based on the temporal rate of change.

In operation 748, the graphics generator 530 determines a color of at least a portion of the graphical representation 300 (e.g., a group of pixels or a region of the graphical representation 300, such as the alert notification illustrated in FIG. 3), and the color of this portion may be determined based on the spatial rate of change in the magnitude of the force (e.g., as determined in operation 630). Accordingly, in example embodiments that include operation 748, the causing of the display screen 120 to present the graphical representation 300 in operation 650 causes the display screen 120 to present the portion whose color is determined based on the spatial rate of change.

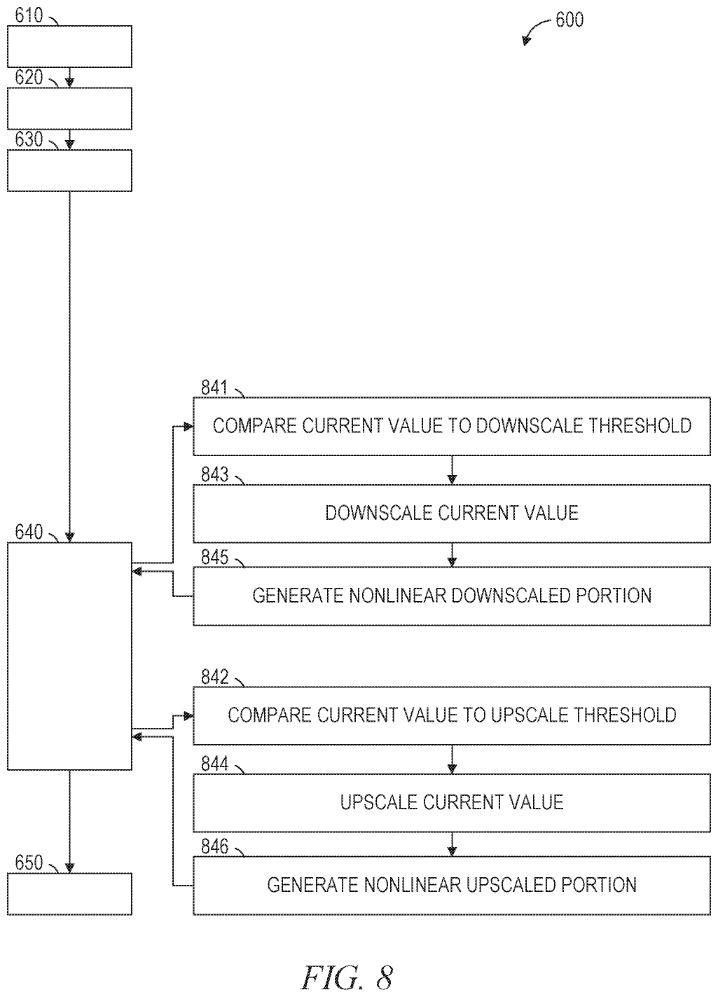

As shown in FIG. 8, in addition to any one or more the operations previously described, the method 600 may include upscaling or downscaling of the current value of the magnitude of the force and graphical depiction thereof by respectively either including operations 841, 843, and 845, or including operations 842, 844, and 846. In either case, the set of included operations may be performed as part of operation 640, in which the graphics generator 530 generates the graphical representation 300 of the force.

In operation 841, the graphics generator 530 compares the current value of the magnitude of the force to a threshold value for the magnitude of the force. The compared threshold value may be a predetermined threshold value (e.g., a predetermined maximum value or a predetermined downscale threshold value).

In operation 843, the graphics generator 530 downscales the current value of the magnitude of the force based on the comparing performed in operation 841. For example, the graphics generator 530 may apply a downscaling coefficient to the current value (e.g., by multiplying the current value by the downscaling coefficient, which may have a value that falls between zero and one) in response to the current value being greater than or equal to the predetermined threshold value compared in operation 841. This may have the effect of introducing nonlinearity to the current magnitude of the magnitude of the force and propagating that nonlinearity into how the current magnitude is represented in the graphical representation 300.

As noted above, according to some example embodiments, a non-linear scaling function is used instead of a comparison to a threshold value. In such example embodiments, operation 841 may be omitted, and operation 843 may instead include calculation of the downscaled value by inputting the current value into the non-linear scaling function (e.g., ƒ(x)=x³) and obtaining the output therefrom.

In operation 845, the graphics generator 530 generates a nonlinear (e.g., scaled down nonlinearly) portion of the graphical representation 300 of the force based on the downscaled current value of the magnitude of the force (e.g., as calculated in operation 843). The resulting graphical representation 300 may accordingly include the generated nonlinear portion, as a result of the current value of the magnitude of the force either transgressing or failing to transgress the predetermined threshold value compared in operation 841.

In operation 842, the graphics generator 530 compares the current value of the magnitude of the force to a threshold value for the magnitude of the force. The compared threshold value may be a predetermined threshold value (e.g., a predetermined minimum value or a predetermined upscale threshold value).

In operation 844, the graphics generator 530 upscales the current value of the magnitude of the force based on the comparing performed in operation 842. For example, the graphics generator 530 may apply an upscaling coefficient to the current value (e.g., by multiplying the current value by the upscaling coefficient, which may have a value greater than one) in response to the current value being less than or equal to the predetermined threshold value compared in operation 842. This may have the effect of introducing nonlinearity to the current magnitude of the magnitude of the force and propagating that nonlinearity into how the current magnitude is represented in the graphical representation 300.

As noted above, according to some example embodiments, a non-linear scaling function is used instead of a comparison to a threshold value. hi such example embodiments, operation 842 may be omitted, and operation 844 may instead include calculation of the upscaled value by inputting the current value into the non-linear scaling function e.g., ƒ(x)=x³) and obtaining the output therefrom.

In operation 846, the graphics generator 530 generates a nonlinear (e.g., scaled up nonlinearly) portion of the graphical representation 300 of the force based on the upscaled current value of the magnitude of the force (e.g., as calculated in operation 844). The resulting graphical representation 300 may accordingly include the generated nonlinear portion, as a result of the current value of the magnitude of the force either transgressing or failing to transgress the predetermined threshold value compared in operation 842.

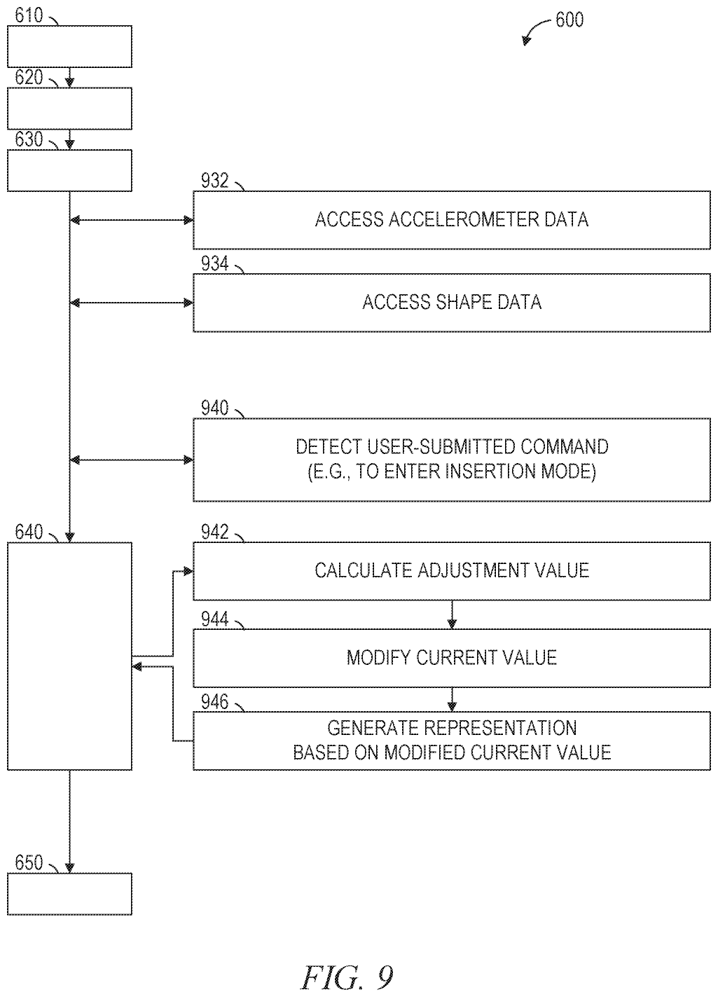

As shown in FIG. 9, in addition to any one or more of the operations previously described, the method 600 may include one or more of operations 932, 934, 940, 942, 944, and 946. One or both of operations 932 and 934 may be performed at any point prior to operation 640, in which the graphics generator 530 generates the graphical representation 300 of the force.

In operation 932, the sensor interface 510 accesses acceleration data generated by the accelerometer 210. As noted above, the accelerometer 210 may be coupled to the elongate device 110 and configured to measure acceleration (e.g., due to gravity, friction, or both) encountered by the elongate device 110 or a portion thereof, in example embodiments that include operation 932, the generation of the graphical representation 300 of the force by the graphics generator 530 in operation 640 is based on the accelerometer data accessed in operation 932.

In operation 934, the sensor interface 510 accesses shape data generated by the shape detector 220. As noted above, the shape detector 220 may be coupled to the elongate device 110 and configured to detect a shape of the elongate device 110 (e.g., detecting positions, orientations, or both of multiple segments of the elongate device 110). In example embodiments that include operation 934, the generation of the graphical representation 300 of the force by the graphics generator 530 in operation 640 is based on the shape data accessed in operation 934.

In operation 940, the sensor interface 510 detects a user-submitted command (e.g., from the operator 140) that the elongate device 110 operate in a. mode (e.g., an insertion mode or a device insertion mode) in which frictional and gravitational forces on at least the distal portion of the elongate device 110 are to be disregarded in generating the graphical representation 300 of the force.

According to various example embodiments, when the elongate device 110 is operating in such a mode, the frictional and gravitational forces are indicated or otherwise determinable (e.g., calculable) based on the temporal rate of change in the magnitude of the force (e.g., as determined in operation 620), the spatial rate of change in the magnitude of the force (e.g., as determined in operation 630), or both, along with potentially one or more additional factors, such as the accelerometer data accessed in operation 932, the shape data accessed in operation 934, position data (e.g., indicating a position of the distal portion of the elongate device 110), velocity data (e.g., indicating a velocity of the distal portion of the elongate device 110), or any suitable combination thereof.

In example embodiments that include operation 940, operations 942, 944, 946 may be performed as part of operation 640, in which the graphics generator 530 generates the graphical representation 300 of the force. in operation 942, the sensor data analyzer 520 calculates an adjustment value by calculating an estimate of cumulative influences (e.g., cumulative forces) from the frictional and gravitational forces acting upon at least the distal portion of the elongate device 110. The adjustment value may be calculated based on the temporal rate of change in the magnitude of the force (e.g., as determined in operation 620), the spatial rate of change in the magnitude of the force (e.g., as determined in operation 630), or both. According to various example embodiments, one or more additional factors contribute to the calculation of the adjustment value, such as the accelerometer data accessed in operation 932, the shape data accessed in operation 934, or both. In certain example embodiments, the adjustment value is calculated as an estimated proportion (e.g., a percentage) of force attributable to the frictional and gravitational threes, rather than an estimated amount (e.g., a value) of the force.

In operation 944, the sensor data analyzer 520 modifies the current value of the magnitude of the force based on the adjustment value calculated in operation 942. For example, the sensor data analyzer 520 may modify the current value by subtracting the adjustment value from the current value. In example embodiments in which the adjustment value is calculated as an estimated percentage of force, the sensor data analyzer 520 modifies the current value by reducing the current value by the estimated percentage (e.g., by multiplying the current value to an intermediate quantity found by subtracting the estimated percentage from unity).

In operation 946, the graphics generator 530 generates the graphical representation 300 of the force based on the modified current value of the magnitude of the force (e.g., as resultant from performance of operation 944). This may be performed in response to the user-submitted command detected in operation 940. Accordingly, in situations where the operator 140 has commanded the control system 100 to operate the elongate device 110 in a mode that disregards frictional and gravitational forces on at least the distal portion of the elongate device 110, the generated and presented graphical representation 300 of the force is accordingly modified (e.g., by using the adjustment value calculated in operation 942) to visually reflect operation in the commanded mode.

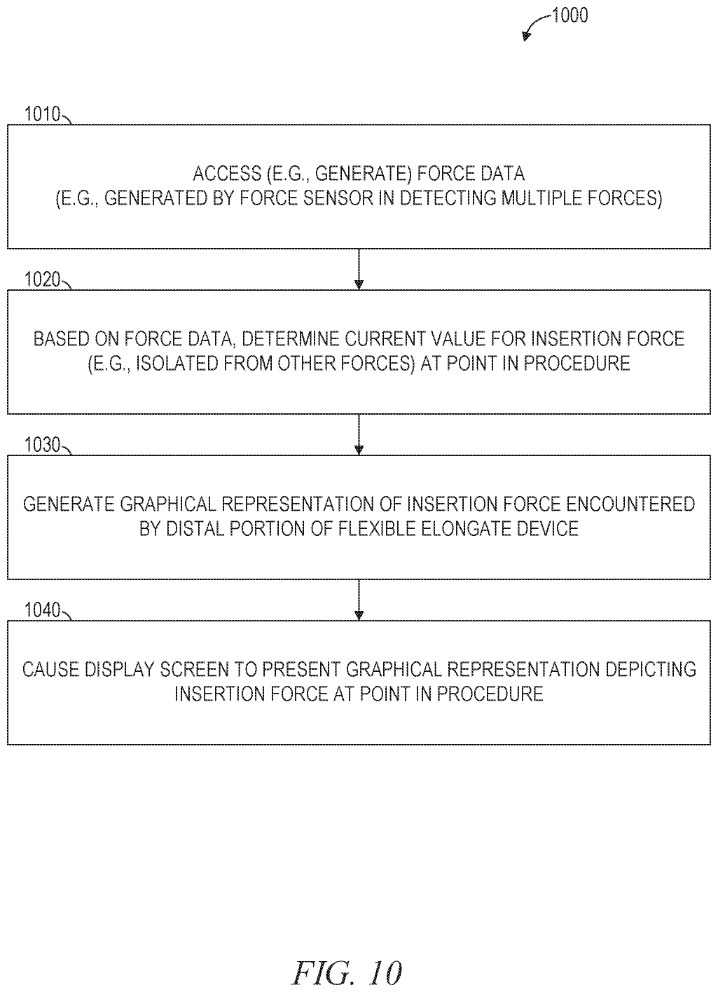

FIGS. 10-12 are flowcharts illustrating operation of the control system 100 (e.g., as described above with respect to FIG. 5, in the context described above with respect to FIGS. 1 and 2, or both) in performing a method 1000 of generating a graphical representation of force (e.g., similar or identical to the graphical representation 300 or visually distinct from the graphical representation 300), according to some example embodiments. Operations in the method 1000 may be performed using components (e.g., modules) described above with respect to FIG. 5, using one or more processors (e.g., microprocessors or other hardware processors), or using any suitable combination thereof. As shown in FIG. 10, the method 1000 includes operations 1010, 1020, 1030, and 1040.

In operation 1010, the sensor interface 510 accesses force data generated by the force sensor 200. In alternative example embodiments, the force sensor 200 generates the three data and directly provides it to the sensor data analyzer 520. The force data quantifies variation in magnitude of a force encountered by the distal portion of the elongate device 110 (e.g., with or without gravitational and frictional forces acting on the entirety of the elongate device 110). This encountered force may estimate, approximate, or otherwise represent an insertion force acting against the distal portion as the distal portion travels (e.g., axially forward) within the environment (e.g., within the anatomy of the patient 130) during performance of a procedure (e.g., a medical procedure, with a predefined workflow from a starting point to an ending point). As noted above, the force sensor 200 is configured to measure the force encountered by the distal portion of the elongate device 110 and then generate the force data based on the measured force. The generated force data accessed in operation 1010 may be live (e.g., substantially real-time) force data and accordingly may include a current value of the magnitude of the force.

In operation 1020, the sensor data analyzer 520 determines the current value of the force, if not readily readable from the force data. In some example embodiments, the sensor data analyzer 520 identifies (e.g., isolates) the current value of the force from contributions made by one or more other threes acting on the elongate device 110 (e.g., gravitational weight, internal friction of components inside the elongate device 110, or both). Thus, the sensor data analyzer 520 obtains the current value of the force (e.g., insertion three, isolated from one or more other forces) at the current point in the procedure relative to the entirety of the procedure (e.g., a current location between starting and ending locations, a current step within a predefined workflow, a current location in the anatomy of the patient, or any suitable combination thereof).

In operation 1030, the graphics generator 530 generates a graphical representation (e.g., similar or identical to the graphical representation 300, or visually distinct from the graphical representation 300) of the force encountered by the distal portion of the elongate device 110. This may be performed in a manner similar to that described above with respect to operation 640 (e.g., by generating all or part of a GUI based on the current value of the magnitude of the force, the temporal rate of change of the force, the spatial rate of change of the force, or any suitable combination thereof). In addition, the generated graphical representation may visually indicate the force (e.g., its current value or a secondary indicator thereof) at a point in the procedure (e.g., the medical procedure) relative to the entirety of the procedure (e.g., at a point relative to the starting point, the ending point, or both).

For example, the generated graphical representation may indicate the force by depicting the current value of the force in a navigational view of the environment (e.g., with a navigational image, as described above with respect to FIG. 1). Accordingly, the operator 140 may view the current value of the force (e.g., insertion force) the same view in which the distal portion of the elongate device 110 is navigated through the environment.

In operation 1040, the graphics generator 530 causes the display screen 120 to present the graphical representation that was generated in operation 1030. This may be performed in a manner similar to that described above with respect to operation 650 (e.g., by providing the generated graphical representation to the display screen 120 as part of a GUI, a video signal, or both. Accordingly, performance of operation 1040 may have the effect of communicating the graphical representation to the operator 140 of the control system 100.

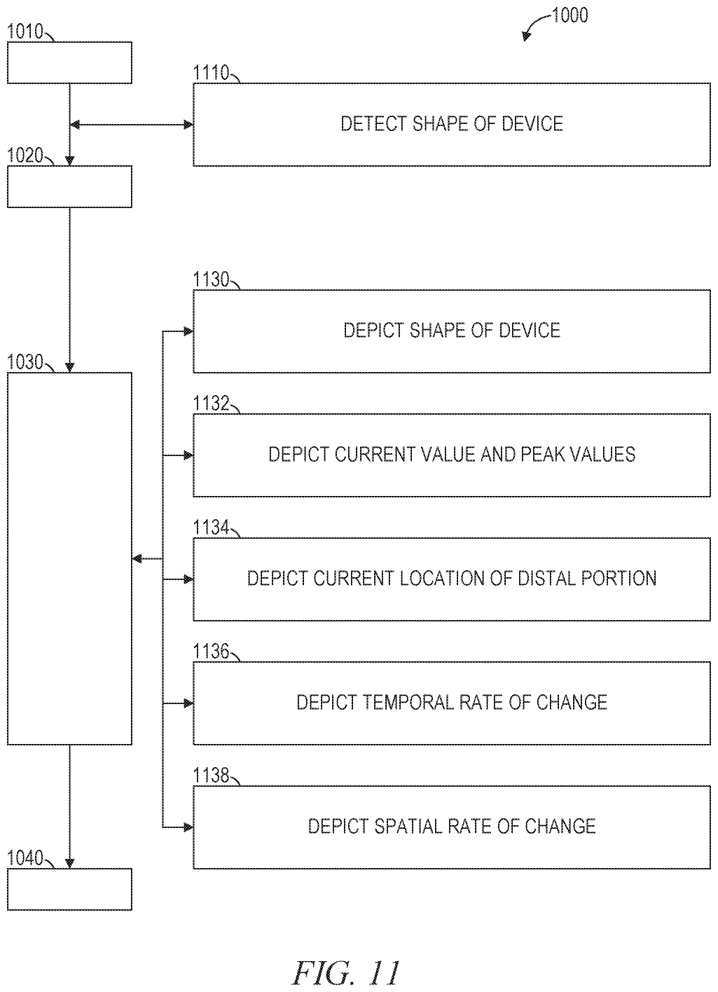

As shown in FIG. 11, in addition to any one or more of the operations previously described for the method 1000, the method 1000 may include one or more of operations 1110, 1130, 1132, 1134, 1136, and 1138.

In operation 1110, the shape detector 220 detects a shape of the elongate device 110 (e.g., detecting positions, orientations, or both of multiple segments of the elongate device 110). This may be performed in a manner similar to that described above with respect to operation 934 in method 600. In example embodiments that include operation 1110, the graphical representation generated in operation 1030 may depict some or all of the detected shape of the elongate device 110.

One or more of operations 1130, 1132, 1134, 1136, and 1138 may be performed as part (e.g., a precursor task, a subroutine, or a portion) operation 1030, in which the graphics generator 530 generates the graphical representation of the force (e.g., insertion force) encountered by the distal portion of the elongate device 110.

In operation 1130, the generating of the graphical representation of the force includes depicting or otherwise causing the generated graphical representation to depict the shape of the elongate device 110 (e.g., as detected in operation 1110). For example, the graphical representation may depict the shape formed by the elongate device 110 and also depict the force (e.g., the current value of the force) encountered by the distal portion of the elongate device 110.

In operation 1132, the generating of the graphical representation of the force includes depicting or otherwise causing the generated graphical representation to depict a current value of the force encountered by the distal portion of the elongate device 110. The current value may be depicted along with one or more peak values of the force (e.g., one or more local maxima in the values of the force thus far in the procedure). Accordingly, the current value may be depicted in the graphical representation relative to (e.g., in proportion to, or as a percentage of) one or more of the peak values (e.g., as a percentage of the maximum peak value, the most recent peak value, or a moving average of the most recent peak values).

In operation 1134, the generating of the graphical representation of the force includes depicting or otherwise causing the generated graphical representation to depict a current location within the environment at which the distal portion of the elongate device 110 (e.g., the distal tip of the elongate device 110) encountered the force being depicted. For example, the graphical representation may depict some or all of the environment (e.g., the anatomy of the patient 130), and the location at which the distal tip of the elongate device 110 encountered the force may be indicated, highlighted, or otherwise shown by the graphical representation.

In operation 1136, the generating of the graphical representation of the force includes depicting or otherwise causing the generated graphical representation to depict a temporal rate of change in the force encountered by the distal portion of the elongate device 110. For example, the temporal rate of change determined in operation 620 of the method 600 may be depicted in the graphical representation. This may have the effect of applying a temporal low-pass filter to any rapid fluctuations in the current value of the force over time, such that the graphical representation of the force is more useful to the operator 140.

In operation 1138, the generating of the graphical representation of the force includes depicting or otherwise causing the generated graphical representation to depict a spatial rate of change in the force encountered by the distal portion of the elongate device 110. For example, the spatial rate of change determined in operation 630 of the method 600 may be depicted in the graphical representation. This may have the effect of applying a spatial low-pass filter to any rapid fluctuations in the current value of the force over distance traveled through the environment, such that the graphical representation of the force is more useful to the operator 140.

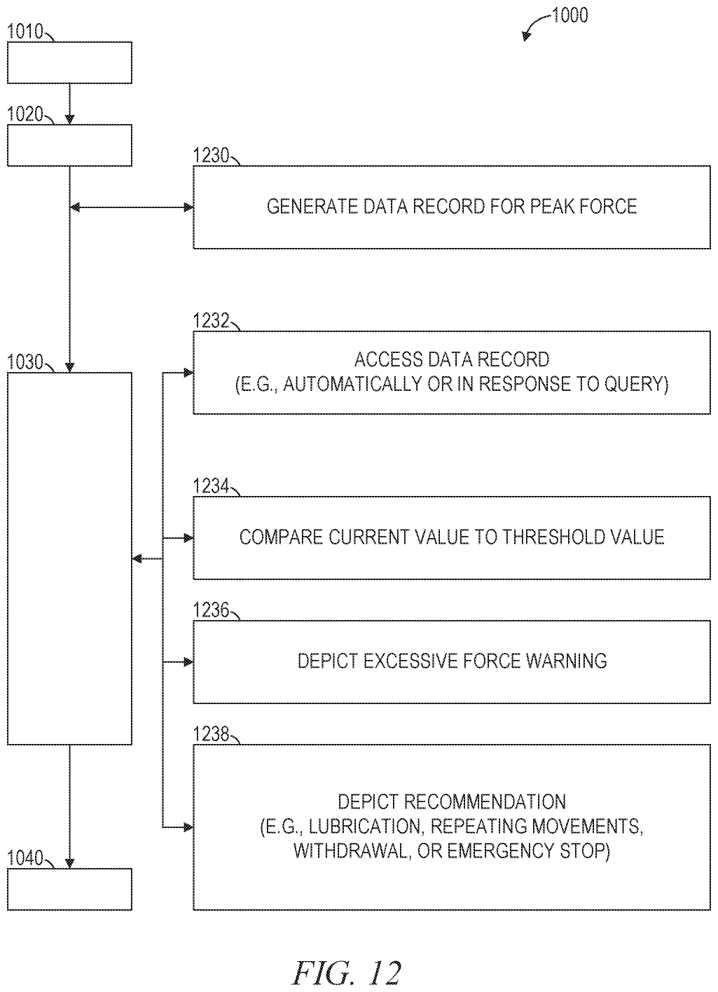

As shown in FIG. 12, in addition to any one or more of the operations previously described for the method 1000, the method 1000 may include one or more of operations 1230, 1232, 1234, 1236, and 1238.

In operation 1230, the sensor data analyzer 520 generates a data record (e.g., within a database included in the control system 100, communicatively coupled thereto, or otherwise accessible by the control system 100). This data record correlates a peak value of the force encountered by the distal portion of the elongate device 110 with the location at which the force was encountered within the environment (e.g., the anatomy of the patient 130)

One or more of operations 1232, 1234, 1236, and 1238 may he performed as part of operation 1030, in which the graphics generator 530 generates the graphical representation of the force (e.g., insertion force) encountered by the distal portion of the elongate device 110.

In operation 1232, the sensor data analyzer 520 accesses the data record generated in operation 1230. This may be performed automatically (e.g., in generating the graphical representation to depict the peak value of the force) or in response to a query (e.g., submitted by the operator 140) to view one or more peak values of the force encountered by the distal portion of the elongate device 110. According to various example embodiments, a performance of operation 1232 may occur at part of operation 1030, at one or more later points in the method 1000 (e.g., after operation 1040 in which the display screen 120 is caused to present the generated graphical representation of the force), or any suitable combination thereof.

In operation 1234, the generating of the graphical representation of the force includes comparing a current value of the force encountered by the distal portion of the elongate device 110 to a threshold value (e.g., a predetermined threshold value of the force, such as a predetermined maximum value for the force). The threshold value may correspond to the environment in which the elongate device 110 is deployed at various levels of specificity. For example, the threshold value may correspond to all lungs of all mammals, all human lungs, all human lungs in a range of ages, or to the lungs of the patient 130 specifically.

In operation 1236, in response to the comparison performed in operation 1234 (e.g., in response to the current value of the force meeting or exceeding the threshold value), the generating of the graphical representation of the force includes depicting or otherwise causing the graphical representation to depict a warning of excessive force. For example, the graphical representation may include an alert or other attention-grabbing indicator that indicates too much force is being applied to or by the distal portion of the elongate device 110.

In operation 1238, in response to the comparison performed in operation 1234 (e.g., in response to the current value of the force meeting or exceeding the threshold value), the generating of the graphical representation of the force includes depicting or otherwise causing the graphical representation to depict a recommendation that the procedure be halted, suspended, or modified. For example, in situations where the distal portion of the elongate device 110 (e.g., its distal tip) has the capability to dispense lubrication, the recommendation may recommend that such a lubrication dispensing operation be inserted into the procedure (e.g., as an additional operation in its workflow). As another example, in situations where encountering a high current value of the force above the threshold value is likely to indicate that stickiness or other cause of high friction is present, the recommendation may recommend that a rapid back-and-forth movement of the distal portion of the elongate device 110 be performed (e.g., automatically or under control by the operator 140) to reduce or otherwise mitigate the friction.

As a further example, in situations where self-lubrication of the distal portion of the elongate device 110 is unavailable or insufficient to address high friction conditions in the environment, the recommendation may recommend that the procedure be suspended, that the distal portion of the elongate device 110 be removed (e.g., withdrawn) from the environment, and that a lubrication operation be performed on some or all of the elongate device 110 prior to reinsertion of the distal portion into the environment and resumption of the procedure. As a still further example, in situations where encountering a high current value of the force above the threshold value is likely to indicate imminent damage to the environment, the elongate device 110, or both, the recommendation may recommend an emergency stop to the procedure (e.g., along with one or more protective or otherwise mitigating actions).

According to various example embodiments, one or more of the systems and methodologies described herein may facilitate generation of the graphical representation 300 of force encountered by the elongate device 110 or a portion (e.g., the distal portion) thereof. Moreover, one or more of the methodologies described herein may facilitate increased awareness of the elongate device 110 with a portion thereof within the environment (e.g., the anatomy of the patient 130) in which the elongate device 110 is deployed. Such increased awareness may include more accurate and more precise awareness of the location, position, orientation, speed, and heading of the distal portion of the elongate device relative to one or more anatomical structures within the anatomy of the patient 130. Hence, one or more of the methodologies described herein may facilitate intuitive control and management of medical instruments, including elongate devices, such as flexible and steerable catheters, that are suitable for performing minimally invasive medical techniques, as well as improved precision and accuracy of performing such medical techniques and the resulting health benefits for patients, compared to capabilities of pre-existing systems and methods.

Additionally, one or more of the systems and methodologies described herein may facilitate generation of a graphical representation of force encountered by the distal portion of the elongate device 110 in relation to an entirety of a procedure being performed using the elongate device 110. Accordingly, one or more of the methodologies discussed herein may facilitate increased awareness of the distal portion of the elongate device 110 and its location within the environment in which it is deployed, as well as the potential for further progress in performing the procedure within the environment and the potential for harming the environment, the elongate device 110, or both. Hence, one or more of the methodologies discussed herein may facilitate increased operational effectiveness, increased patient safety, increased patient comfort, reduced recovery time, reduced mechanical wear on the elongate device 110, reduced risk of damage to the elongate device 110, or any suitable combination thereof.

When these effects are considered in aggregate, one or more of the systems and methodologies described herein may obviate a need for certain efforts or resources that otherwise would be involved in generation of a graphical representation of force encountered by a device. Efforts expended by a user (e.g., the operator 140) in estimating or otherwise interpreting how much force is being encountered by the elongate device 110 may be reduced by use of (e.g., reliance upon) a special-purpose machine that implements one or more of the methodologies described herein. Computing resources used by one or more systems or machines may similarly be reduced (e.g., compared to systems or machines that lack the structures discussed herein or are otherwise unable to perform the functions discussed herein). Examples of such computing resources include processor cycles, network traffic, computational capacity, main memory usage, graphics rendering capacity, graphics memory usage, data storage capacity, power consumption, and cooling capacity.

FIG. 13 is a block diagram illustrating components of a machine 1300, according to some example embodiments, able to read instructions 1324 from a machine-readable medium 1322 (e.g., a non-transitory machine-readable medium, a machine-readable storage medium, a computer-readable storage medium, or any suitable combination thereof) and perform any one or more of the methodologies discussed herein, in whole or in part. Specifically, FIG. 13 shows the machine 1300 in the example form of a computer system (e.g., a. computer) within which the instructions 1324 (e.g., software, a program, an application, an applet, an app, or other executable code) for causing the machine 1300 to perform any one or more of the methodologies discussed herein may be executed, in whole or in part.

In alternative embodiments, the machine 1300 operates as a standalone device or may be communicatively coupled (e.g., networked) to other machines. In a networked deployment, the machine 1300 may operate in the capacity of a server machine or a client machine in a server-client network environment, or as a peer machine in a distributed (e.g., peer-to-peer) network environment. The machine 1300 may be a server computer, a client computer, a personal computer (PC), a tablet computer, a laptop computer, a netbook, a cellular telephone, a smart phone, a set-top box (STB), a personal digital assistant (PDA), a web appliance, a network router, a network switch, a network bridge, or any machine capable of executing the instructions 1324, sequentially or otherwise, that specify actions to be taken by that machine. Further, while only a single machine is illustrated, the term “machine” shall also be taken to include any collection of machines that individually or jointly execute the instructions 1324 to perform all or part of any one or more of the methodologies discussed herein.

The machine 1300 includes a processor 1302 (e.g., one or more central processing units (CPUs), one or more graphics processing units (CPUs), one or more digital signal processors (DSPs), one or more application specific integrated circuits (ASICs), one or more radio-frequency integrated circuits (RFICs), or any suitable combination thereof), a main memory 1304, and a static memory 1306, which are configured to communicate with each other via a bus 1308. The processor 1302 contains solid-state digital microcircuits (e.g., electronic, optical, or both) that are configurable, temporarily or permanently, by some or all of the instructions 1324 such that the processor 1302 is configurable to perform any one or more of the methodologies described herein, in whole or in part. For example, a set of one or more microcircuits of the processor 1302 may be configurable to execute one or more modules (e.g., software modules) described herein. In some example embodiments, the processor 1302 is a multicore CPU (e.g., a dual-core CPU, a quad-core CPU, an 8-core CPU, or a 128-core CPU) within which each of multiple cores behaves as a separate processor that is able to perform any one or more of the methodologies discussed herein, in whole or in part. Although the beneficial effects described herein may be provided by the machine 1300 with at least the processor 1302, these same beneficial effects may be provided by a different kind of machine that contains no processors (e.g., a purely mechanical system, a purely hydraulic system, or a hybrid mechanical-hydraulic system), if such a processor-less machine is configured to perform one or more of the methodologies described herein.

The machine 1300 may further include a graphics display 1310 (e.g., a plasma display panel (PDP), a light emitting diode (LED) display, a liquid crystal display (LCD), a projector, a cathode ray tube (CRT), or any other display capable of displaying graphics or video). The machine 1300 may also include an alphanumeric input device 1312 (e.g., a keyboard or keypad), a pointer input device 1314 (e.g., a mouse, a touchpad, a touchscreen, a trackball, a joystick, a stylus, a motion sensor, an eye tracking device, a data glove, or other pointing instrument), a data storage 1316, an audio generation device 1318 a sound card, an amplifier, a speaker, a headphone jack, or any suitable combination thereof), and a network interface device 1320.

The data storage 1316 (e.g., a data storage device) includes the machine-readable medium 1322 (e.g., a tangible and non-transitory machine-readable storage medium) on which are stored the instructions 1324 embodying any one or more of the methodologies or functions described herein. The instructions 1324 may also reside, completely or at least partially, within the main memory 1304, within the static memory 1306, within the processor 1302 (e.g., within the processor's cache memory), or any suitable combination thereof, before or during execution thereof by the machine 1300. Accordingly, the main memory 1304, the static memory 1306, and the processor 1302 may be considered machine-readable media (e.g., tangible and non-transitory machine-readable media). The instructions 1324 may be transmitted or received over the network 190 via the network interface device 1320. For example, the network interface device 1320 may communicate the instructions 1324 using any one or more transfer protocols (e.g., hypertext transfer protocol (HTTP)).

In some example embodiments, the machine 1300 may be a portable computing device (e.g., a smart phone, a tablet computer, or a wearable device), and may have one or more additional input components 1330 (e.g., sensors or gauges). Examples of such input components 1330 include an image input component (e.g., one or more cameras), an audio input component (e.g., one or more microphones), a direction input component (e.g., a compass), a location input component (e.g., a global positioning system (GPS) receiver), an orientation component (e.g., a gyroscope), a motion detection component (e.g., one or more accelerometers), an altitude detection component (e.g., an altimeter), a temperature input component (e.g., a thermometer), and a gas detection component a gas sensor). Input data gathered by any one or more of these input components 1330 may be accessible and available for use by any of the modules described herein (e.g., with suitable privacy notifications and protections, such as opt-in consent or opt-out consent, implemented in accordance with user preference, applicable regulations, or any suitable combination thereof).

As used herein, the term “memory” refers to a machine-readable medium able to store data temporarily or permanently and may be taken to include, but not be limited to, random-access memory (RAM), read-only memory (ROM), buffer memory, flash memory, and cache memory. While the machine-readable medium 1322 is shown in an example embodiment to be a single medium, the term “machine-readable medium” should be taken to include a single medium or multiple media (e.g., a centralized or distributed database, or associated caches and servers) able to store instructions. The term “machine-readable medium” shall also be taken to include any medium, or combination of multiple media, that is capable of carrying (e.g., storing or communicating) the instructions 1324 for execution by the machine 1300, such that the instructions 1324, when executed by one or more processors of the machine 1300 (e.g., processor 1302), cause the machine 1300 to perform any one or more of the methodologies described herein, in whole or in part. Accordingly, a “machine-readable medium” refers to a single storage apparatus or device, as well as cloud-based storage systems or storage networks that include multiple storage apparatus or devices. The term “machine-readable medium” shall accordingly be taken to include, but not be limited to, one or more tangible and non-transitory data repositories (e.g., data volumes) in the example form of a solid-state memory chip, an optical disc, a magnetic disc, or any suitable combination thereof.

A “non-transitory” machine-readable medium, as used herein, specifically excludes propagating signals per se. According to various example embodiments, the instructions 1324 for execution by the machine 1300 can be communicated via a carrier medium (e.g., a machine-readable carrier medium). Examples of such a carrier medium include a non-transient carrier medium (e.g., a non-transitory machine-readable storage medium, such as a solid-state memory that is physically movable from one place to another place) and a transient carrier medium (e.g., a carrier wave or other propagating signal that communicates the instructions 1324).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions described herein. As used herein, “processor-implemented module” refers to a hardware module in which the hardware includes one or more processors. Accordingly, the operations described herein may be at least partially processor-implemented, hardware-implemented, or both, since a processor is an example of hardware, and at least some operations within any one or more of the methods discussed herein may be performed by one or more processor-implemented modules, hardware-implemented modules, or any suitable combination thereof.

Moreover, such one or more processors may perform operations in a “cloud computing” environment or as a service (e.g., within a “software as a service” (SaaS) implementation). For example, at least some operations within any one or more of the methods discussed herein may be performed by a group of computers (e.g., as examples of machines that include processors), with these operations being accessible via a network (e.g., the Internet) and via one or more appropriate interfaces (e.g., an application program interface (API)). The performance of certain operations may be distributed among the one or more processors, whether residing only within a single machine or deployed across a number of machines. In some example embodiments, the one or more processors or hardware modules (e.g., processor-implemented modules) may be located in a single geographic location (e.g., within a home environment, an office environment, or a server farm). In other example embodiments, the one or more processors or hardware modules may be distributed across a number of geographic locations.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and their functionality presented as separate components and functions in example configurations may be implemented as a combined structure or component with combined functions. Similarly, structures and functionality presented as a single component may be implemented as separate components and functions. These and other variations, modifications, additions, and improvements fall within the scope of the subject matter herein.

Some portions of the subject matter discussed herein may be presented in terms of algorithms or symbolic representations of operations on data stored as bits or binary digital signals within a memory (e.g., a computer memory or other machine memory). Such algorithms or symbolic representations are examples of techniques used by those of ordinary skill in the data processing arts to convey the substance of their work to others skilled in the art. As used herein, an “algorithm” is a self-consistent sequence of operations or similar processing leading to a desired result. In this context, algorithms and operations involve physical manipulation of physical quantities. Typically, but not necessarily, such quantities may take the form of electrical, magnetic, or optical signals capable of being stored, accessed, transferred, combined, compared, or otherwise manipulated by a machine. It is convenient at times, principally for reasons of common usage, to refer to such signals using words such as “data,” “content,” “bits,” “values,” “elements,” “symbols,” “characters,” “terms,” “numbers,” “numerals,” or the like. These words, however, are merely convenient labels and are to be associated with appropriate physical quantities.

Unless specifically stated otherwise, discussions herein using words such as “accessing,” “processing,” “detecting,” “computing,” “calculating,” “determining,” “generating,” “presenting,” “displaying,” or the like refer to actions or processes performable by a machine (e.g., a computer) that manipulates or transforms data represented as physical (e.g., electronic, magnetic, or optical) quantities within one or more memories (e.g., volatile memory, non-volatile memory, or any suitable combination thereof), registers, or other machine components that receive, store, transmit, or display information. Furthermore, unless specifically stated otherwise, the terms “a” or “an” are herein used, as is common in patent documents, to include one or more than one instance. Finally, as used herein, the conjunction “or” refers to a non-exclusive “or,” unless specifically stated otherwise.

The following enumerated descriptions describe various examples of methods, machine-readable media, and systems e.g., machines, devices, or other apparatus) discussed herein.

A first example provides a system comprising:
an elongate device (e.g., a flexible elongate device) including a distal portion configured to travel within an environment (e.g., and including a proximal portion configured to remain external to the environment);
a force sensor coupled to the elongate device (e.g., coupled to the proximal portion thereof) and configured to detect (e.g., measure) a force (e.g., a force encountered by the distal portion of the elongate device during a time period in which the distal portion travels a distance within the environment), the force sensor being configured to generate force data based or detected (e.g., measured) force;
a display screen;
one or more processors; and
a memory storing instructions that, when executed by at least one processor among the one or more processors, cause the at least one processor to perform operations comprising:
accessing the force data generated by the force sensor, the force data quantifying variation in magnitude of the force encountered by the distal portion of the elongate device during the time period in which the distal portion traveled the distance within the environment, the force data including a current value of the magnitude of the force;
determining a temporal rate of change of the force during the time period based on the force data and the time period;
determining a spatial rate of change of the force based on the force data and the distance traveled by the distal portion of the elongate device;
generating a graphical representation (e.g., a real-time graphical representation) of the force based on the current value of the magnitude of the force, the temporal rate of change of the force, and the spatial rate of change of the force; and
causing the display screen to present the graphical representation generated based on the current value, the temporal rate of change, and the spatial rate of change.

A second example provides a system according to the first example, wherein:
the determining of the temporal rate of change of the force includes calculating a ratio of the variation in the magnitude of the force to a duration of the time period in which the distal portion of the elongate device traveled the distance within the environment; and
the generating of the graphical representation of the force is based on the ratio of the variation in the magnitude of the force to the duration of the time period.

A third example provides a system according to the first example or the second example, wherein:
the determining of the spatial rate of change of the force includes calculating a ratio of the variation in the magnitude of the force to the distance traveled by the distal portion of the elongate device within the environment; and
the generating of the graphical representation of the force is based on the ratio of the variation in the magnitude of the force to the distance traveled by the distal portion.

A fourth example provides a system according to any of the first through third examples, wherein:
the generating of the graphical representation of the force includes determining a color of at least a portion of the graphical representation based on the temporal rate of change of the force; and
the causing of the display screen to present the graphical representation includes causing the display screen to present the portion whose color is determined based on the temporal rate of change of the force.

A fifth example provides a system according to any of the first through fourth examples, wherein:
the generating of the graphical representation of the force includes determining a color of at least a portion of the graphical representation based on the spatial rate of change of the force; and
the causing of the display screen to present the graphical representation includes causing the display screen to present the portion whose color is determined based on the spatial rate of change of the force.

A sixth example provides a system according to any of the first through fifth examples, wherein the operations further comprise:
weighting the current value of the magnitude of the force, the temporal rate of change of the force, and the spatial rate of change of the force; and wherein:
the generating of the graphical representation of the force is based on the weighted current value, the weighted temporal rate of change, and the weighted spatial rate of change.

A seventh example provides a system according to the sixth example, wherein the operations further comprise:
determining a set of weighting coefficients for the current value of the magnitude of the force, the temporal rate of change of the force, and the spatial rate of change of the force; and wherein:
the weighting of the current value of the force, the temporal rate of change of the force, and the spatial rate of change of the force is based on the determined set of weighting coefficients.

An eighth example provides a system according to the seventh example, wherein:
the determining of the set of weighting coefficients includes detecting a user-submitted command that specifies the set of weighting coefficients; and
the weighting of the current value of the force, the temporal rate of change of the force, and the spatial rate of change of the force is based on the set of weighting coefficients specified by the user-submitted command.

A ninth example provides a system according to the seventh example, wherein:
the determining of the set of weighting coefficients includes selecting a predetermined set of weighting coefficients from a plurality of predetermined sets of weighting coefficients; and
the weighting of the current value of the force, the temporal rate of change of the force, and the spatial rate of change of the force is based on the predetermined set selected from the plurality of predetermined sets.

A tenth example provides a system according to the ninth example. wherein:
each predetermined set of weighting coefficients in the plurality of predetermined sets of weighting coefficients corresponds to a different combination of human health conditions.

An eleventh example provides a system according to any of the first through tenth examples, wherein:
the generating of the graphical representation of the force includes:
comparing the current value of the magnitude of the force to a threshold value;
downscaling the current value of the magnitude of the force based on the comparing; and
generating a nonlinear portion of the graphical representation of the force, the nonlinear portion depicting the downscaled current value of the magnitude of the force.

A twelfth example provides a system according to any of the first through tenth examples, wherein:
the generating of the graphical representation of the force includes:
comparing the current value of the magnitude of the force to a threshold value;
upscaling the current value of the magnitude of the force based on the comparing; and
generating a nonlinear portion of the graphical representation of the force, the nonlinear portion depicting the upscaled current value of the magnitude of the force.

A thirteenth example provides a system according to any of the first through twelfth examples, further comprising:
an accelerometer coupled to the elongate device and configured to detect (e.g., measure) an acceleration encountered by the elongate device during the time period in which the distal portion travels the distance within the environment, the accelerometer being configured to generate acceleration data based on the detected (e.g., measured) acceleration; wherein
the operations further comprise:
accessing the acceleration data generated by the accelerometer; and wherein
the generating of the graphical representation of the force is based on the accelerometer data.

A fourteenth example provides a system according to any of the first through thirteenth examples, further comprising:
a shape detector coupled to the elongate device and configured to detect a shape of the elongate device during the time period in which the distal portion travels the distance within the environment, the shape detector being configured to generate shape data based on the detected shape; wherein
the operations further comprise:
accessing the shape data generated by the shape detector; and wherein
the generating of the graphical representation of the force is based on the shape data.

A fifteenth example provides a system according to any of the first through fourteenth examples, wherein the operations further comprise:
detecting a user-submitted command that the elongate device operate in a device insertion mode in which frictional and gravitational forces on the distal portion of the elongate device are to be disregarded; and wherein:
the generating of the graphical representation of the force includes:
calculating an adjustment value that estimates influences from frictional and gravitational forces on the distal portion of the elongate device, the adjustment value being calculated based on the temporal and spatial rates of change of the force;
modifying the current value of the magnitude of the force by subtracting the calculated adjustment value therefrom; and
generating the graphical representation of the force based on the modified current value of the magnitude of the force in response to the user-submitted command that the elongate device operate in the insertion mode.

A sixteenth example provides a system according to any of the first through fifteenth examples, wherein:
the environment in which the distal portion of the elongate device travels the distance is an anatomy of a human patient;
the elongate device is a flexible robotic surgical catheter that has an axial direction from a proximal portion of the flexible robotic surgical catheter to the distal portion of the flexible robotic surgical catheter;
the force encountered by the distal portion of the flexible robotic surgical catheter is detected (e.g., measured) by an axial force sensor during the time period in which the distal portion travels the distance within the anatomy of the human patient; and
the force data is generated by the axial force sensor and quantifies variation in the magnitude of the axial force applied to the distal portion of the flexible robotic surgical catheter against its axial direction.

A seventeenth example provides a method comprising:
accessing, by one or more processors, force data generated by a force sensor, the force data quantifying variation in magnitude of a force encountered by a distal portion of an elongate device during a time period in which the distal portion traveled a distance within an environment, the force data including a current value of the magnitude of the force;
determining, by one or more of the processors, a temporal rate of change of the force during the time period based on the force data and the time period;
determining, by one or more of the processors, a spatial rate of change of the force based on the force data and the distance traveled by the distal portion of the elongate device;
generating, by one or more of the processors, a graphical representation (e.g., a real-time graphical representation) of the force based on the current value of the magnitude of the force, the temporal rate of change of the force, and the spatial rate of change of the three; and
causing, by one or more of the processors, a display screen to present the graphical representation generated based on the current value, the temporal rate of change, and the spatial rate of change.

An eighteenth example provides a method according to the seventeenth example, wherein:
the generating of the graphical representation of the force includes determining a color of at least a portion of the graphical representation based on the spatial rate of change of the force; and
the causing of the display screen to present the graphical representation includes causing the display screen to present the portion whose color is determined based on the spatial rate of change of the force.

A nineteenth example provides a machine-readable medium (e.g., a non-transitory machine-readable storage medium) comprising instructions that, when executed by one or more processors of a machine, cause the machine to perform operations comprising:
accessing force data generated by a force sensor, the force data quantifying variation in magnitude of a force encountered by a distal portion of an elongate device during a time period in which the distal portion traveled a distance within an environment, the force data including a current value of the magnitude of the force:
determining a temporal rate of change of the force during the time period based on the force data and the time period;
determining a spatial rate of change of the force based on the force data and the distance traveled by the distal portion of the elongate device;
generating a graphical representation (e.g., a real-time graphical representation) of the force based on the current value of the magnitude of the force, the temporal rate of change of the force, and the spatial rate of change of the force; and
causing a display screen to present the graphical representation generated based on the current value, the temporal rate of change, and the spatial rate of change.

A twentieth example provides a machine-readable medium according to the nineteenth example, wherein:
the determining of the spatial rate of change of the force includes calculating a ratio of the variation in the magnitude of the force to the distance traveled by the distal portion of the elongate device within the environment; and
the generating of the graphical representation of the force is based on the ratio of the variation in the magnitude of the force to the distance traveled by the distal portion.

A twenty-first example provides a machine-readable medium according to the nineteenth example, wherein:
the generating of the graphical representation resource includes:
generating a graphical bar among a sequence of graphical bars, the sequence having an axis, the generated graphical bar having a cross-axis length that represents the current value of the magnitude of the force and an axis-aligned length that represents the time period in which the distal portion traveled the distance within the environment.

A twenty-second example provides a system comprising:
a flexible elongate device including a distal portion configured to travel within an environment and a proximal portion configured to remain external to the environment;
a force sensor coupled to the proximal portion of the flexible elongate device and configured to generate force data during a procedure;
a control system communicatively coupled to the flexible elongate device and the force sensor, the control system being configured to, based on the force data, generate a graphical representation that indicates a force encountered by the distal portion of the flexible elongate device at a point in the procedure relative to an entirety of the procedure; and
a display screen communicatively coupled to the control system and configured to present the graphical representation indicative of the force encountered by the distal portion at the point relative to the entirety of the procedure.

A twenty-third example provides a system according to the twenty-second example, fluffier comprising:
a shape detector configured to detect a shape formed by the flexible elongate device; and wherein:
the graphical representation depicts the three encountered by the distal portion of the flexible elongate device in the detected shape formed by the flexible elongate device within the environment.

A twenty-fourth example provides a system according to the twenty-second example or the twenty-third example, wherein:
the graphical representation depicts a current value of the force encountered by the distal portion of the flexible elongate device relative to one or more peak values of the force (e.g., encountered by the distal portion of the flexible elongate device during the procedure)

A twenty-fifth example provides a system according to any one of the twenty-second through twenty-fourth examples, wherein:
the graphical representation depicts a current location in the environment and at which the distal portion of the flexible elongate device encountered the indicated force.

A twenty-sixth example provides a system according to any one of the twenty-second through twenty-fifth examples, wherein:
the control system is configured to generate a data record that correlates a peak value of the force encountered by the distal portion of the flexible elongate device with a location at which the distal portion of the flexible elongate device encountered the peak value of the force; and
the control system is further configured to access the generated record in response to a query for locations at which peak values of the force occurred during the procedure.

A twenty-seventh example provides a system according to any one of the twenty-second through twenty-sixth examples, wherein:
the graphical representation depicts a temporal rate of change in the force encountered by the distal portion of the flexible elongate device.

A twenty-eighth example provides a system according to any one of the twenty-second through twenty-seventh examples, wherein:
the graphical representation depicts a spatial rate of change in the force encountered by the distal portion of the flexible elongate device.

A twenty-ninth example provides a system according to any one of the twenty-second through twenty-eighth examples, wherein:
the control system is configured to compare a current value of the force encountered by the distal portion of the flexible elongate device to a threshold value that corresponds to the environment; and
based on the current value exceeding the threshold value, the graphical representation depicts a warning that the current value of the force exceeds the threshold value.

A thirtieth example provides a system according to any one of the twenty-second through twenty-ninth examples, wherein:
the control system is configured to compare a current value of the force encountered by the distal portion of the flexible elongate device to a threshold value that corresponds to the environment; and
based on the current value exceeding the threshold value, the graphical representation depicts a recommendation that the procedure be modified.

A thirty-first example provides a system according to the thirtieth example, wherein:
the recommendation depicted in the graphical representation recommends at least one of:
a lubrication dispensing operation by the distal portion of the flexible elongate device,
a set of repeated movements by the distal portion of the flexible elongate device;
a removal of the distal portion of the flexible elongate device from the environment followed by lubrication of the distal portion; or
an emergency stop of the procedure.

A thirty-second example provides a method comprising:
generating, by one or more processors, force data based on input from a force sensor communicatively coupled to a proximal portion of a flexible elongate device during a procedure in which a distal portion of the flexible elongate device travels within an environment;
generating, based on the force data and by one or more of the processors, a graphical representation that indicates a force encountered by a distal portion of the flexible elongate device at a point in the procedure relative to an entirety of the procedure; and
causing, by one or more of the processors, a display screen to present the graphical representation indicative of the force encountered by the distal portion at the point relative to the entirety of the procedure.

A thirty-third example provides a method according to the thirty-second example, further comprising:
detecting a shape formed by the flexible elongate device within the environment; and wherein:
the graphical representation depicts the force encountered by the distal portion of the flexible elongate device in the detected shape formed by the flexible elongate device.

A thirty-fourth example provides a method according to the thirty-second example or the thirty-third example, wherein:
the graphical representation depicts a current value of the force encountered by the distal portion of the flexible elongate device relative to one or more peak values of the force (e.g., encountered by the distal portion of the flexible elongate device during the procedure).

A thirty-fifth example provides a method according to any of the thirty-second through thirty-fourth examples, wherein:
the graphical representation depicts a current location in the environment and at which the distal portion of the flexible elongate device encountered the indicated force.

A thirty-sixth example provides a method according to any of the thirty-second through thirty-fifth examples, further comprising:
generating a data record that correlates a peak value of the force encountered by the distal portion of the flexible elongate device with a location at which the distal portion of the flexible elongate device encountered the peak value of the force; and
accessing the generated record in response to a query for locations at which peak values of the force occurred during the procedure.

A thirty-seventh example provides a method according to any of the thirty-second through thirty-sixth examples, wherein:
the graphical representation depicts a temporal rate of change in the force encountered by the distal portion of the flexible elongate device.

A thirty-eighth example provides a method according to any of the thirty-second through thirty-seventh examples, further comprising:
comparing a current value of the force encountered by the distal portion of the flexible elongate device to a threshold value that corresponds to the environment; and wherein:
based on the current value exceeding the threshold value, the graphical representation depicts a warning that the current value of the force exceeds the threshold value.

A thirty-ninth example provides a method according to any of the thirty-second through thirty-eighth examples, further comprising:
comparing a current value of the force encountered by the distal portion of the flexible elongate device to a threshold value that corresponds to the environment; and wherein:
based on the current value exceeding the threshold value, the graphical representation depicts a recommendation that the procedure be modified.

A fortieth example provides a method according to the thirty-ninth example, wherein:
the recommendation depicted in the graphical representation recommends at least one of:
a lubrication dispensing operation by the distal portion of the flexible elongate device,
a set of repeated movements by the distal portion of the flexible elongate device;
a removal of the distal portion of the flexible elongate device from the environment followed by lubrication of the distal portion; or
an emergency stop of the procedure.

A forty-first example provides a machine-readable medium (e.g., a non-transitory machine-readable storage medium) comprising instructions that, when executed by one or more processors of a machine, cause the machine to perform operations comprising:
generating force data based on input from a three sensor communicatively coupled to a proximal portion of a flexible elongate device during a procedure in which a distal portion of the flexible elongate device travels within an environment;
generating, based on the force data, a graphical representation that indicates a force encountered by a distal portion of the flexible elongate device at a point in the procedure relative to an entirety of the procedure; and
causing a display screen to present the graphical representation indicative of the force encountered by the distal portion at the point relative to the entirety of the procedure.

A forty-second example provides a carrier medium carrying machine-readable instructions for controlling a machine to carry out the operations (e.g., method operations) performed in any one of the previously described examples.

What is claimed is:

1. A system comprising:
a flexible elongate device including a distal portion configured to travel within an environment and a proximal portion configured to remain external to the environment;
a force sensor coupled to the proximal portion of the flexible elongate device and configured to generate force data during a procedure;
a control system communicatively coupled to the flexible elongate device and the force sensor, the control system being configured to, based on the force data, generate a graphical representation that indicates a force encountered by the distal portion of the flexible elongate device at a point in the procedure relative to an entirety of the procedure; and
a display screen communicatively coupled to the control system and configured to present the graphical representation indicative of the force encountered by the distal portion at the point relative to the entirety of the procedure.

2. The system of claim 1, further comprising:
a shape detector configured to detect a shape formed by the flexible elongate device; and wherein:
the graphical representation depicts the force encountered by the distal portion of the flexible elongate device in the detected shape formed by the flexible elongate device within the environment.

3. The system of claim 1, wherein:
the graphical representation depicts a current value of the force encountered by the distal portion of the flexible elongate device relative to one or more peak values of the force.

4. The system of claim 1, wherein:
the graphical representation depicts a current location in the environment and at which the distal portion of the flexible elongate device encountered the indicated force,

5. The system of claim 1, wherein:
the control system is configured to generate a data record that correlates a peak value of the force encountered by the distal portion of the flexible elongate device with a location at which the distal portion of the flexible elongate device encountered the peak value of the force; and
the control system is further configured to access the generated record in response to a query for locations at which peak values of the force occurred during the procedure.

6. The system of claim 1, wherein:
the graphical representation depicts a temporal rate of change in the force encountered by the distal portion of the flexible elongate device,

7. The system of claim 1, wherein:
the graphical representation depicts a spatial rate of change in the force encountered by the distal portion of the flexible elongate device.

8. The system of claim 1, wherein:
the control system is configured to compare a current value of the force encountered by the distal portion of the flexible elongate device to a threshold value that corresponds to the environment; and
based on the current value exceeding the threshold value, the graphical representation depicts a warning that the current value of the force exceeds the threshold value.

9. The system of claim 1, wherein:
the control system is configured to compare a current value of the force encountered by the distal portion of the flexible elongate device to a threshold value that corresponds to the environment; and
based on the current value exceeding the threshold value, the graphical representation depicts a recommendation that the procedure be modified.

10. The system of claim 9, wherein:
the recommendation depicted in the graphical representation recommends at least one of
a lubrication dispensing operation by the distal portion of the flexible elongate device,
a set of repeated movements by the distal portion of the flexible elongate device;
a removal of the distal portion of the flexible elongate device from the environment followed by lubrication of the distal portion; or
an emergency stop of the procedure.

11. A machine-readable storage medium comprising instructions that, when executed by one or more processors of a machine, cause the machine to perform operations comprising:
generating force data based on input from a force sensor communicatively coupled to a proximal portion of a flexible elongate device during a procedure in which a distal portion of the flexible elongate device travels within an environment;
generating, based on the force data, a graphical representation that indicates a force encountered by a distal portion of the flexible elongate device at a point in the procedure relative to an entirety of the procedure; and
causing a display screen to present the graphical representation indicative of the force encountered by the distal portion at the point relative to the entirety of the procedure.

12. The machine-readable storage medium of claim 11, wherein the operations further comprise:
detecting a shape formed by the flexible elongate device within the environment; and wherein:
the graphical representation depicts the force encountered by the distal portion of the flexible elongate device in the detected shape formed by the flexible elongate device.

13. The machine-readable storage medium of claim 11, wherein:
the graphical representation depicts a current value of the force encountered by the distal portion of the flexible elongate device relative to one or more peak values of the force.

14. The machine-readable storage medium of claim 11, wherein:
the graphical representation depicts a current location in the environment and at which the distal portion of the flexible elongate device encountered the indicated force.

15. The machine-readable storage medium of claim 11, wherein the operations further comprise:
generating a data record that correlates a peak value of the force encountered by the distal portion of the flexible elongate device with a location at which the distal portion of the flexible elongate device encountered the peak value of the force; and
accessing the generated record in response to a query for locations at which peak values of the force occurred during the procedure.

16. The machine-readable storage medium of claim 11, wherein:
the graphical representation depicts a temporal rate of change in the force encountered by the distal portion of the flexible elongate device.

17. The machine-readable storage medium of claim 11, wherein the operations further comprise:
comparing a current value of the force encountered by the distal portion of the flexible elongate device to a threshold value that corresponds to the environment; and wherein:
based on the current value exceeding the threshold value, the graphical representation depicts a warning that the current value of the force exceeds the threshold value.

18. The machine-readable storage medium of claim 11, wherein the operations further comprise:
comparing a current value of the force encountered by the distal portion of the flexible elongate device to a threshold value that corresponds to the environment; and wherein:
based on the current value exceeding the threshold value, the graphical representation depicts a recommendation that the procedure be modified.

19. The machine-readable storage medium of claim 18, wherein:
the recommendation depicted in the graphical representation recommends at least one of:
a lubrication dispensing operation by the distal portion of the flexible elongate device,
a set of repeated movements by the distal portion of the flexible elongate device;
a removal of the distal portion of the flexible elongate device from the environment followed by lubrication of the distal portion; or
an emergency stop of the procedure.

20. A method comprising:
generating, by one or more processors, force data based on input from a force sensor communicatively coupled to a proximal portion of a flexible elongate device during a procedure in which a distal portion of the flexible elongate device travels within an environment:
generating, based on the force data and by one or more of the processors, a graphical representation that indicates a force encountered by a distal portion of the flexible elongate device at a point in the procedure relative to an entirety of the procedure; and
causing, by one or more of the processors, a display screen to present the graphical representation indicative of the force encountered by the distal portion at the point relative to the entirety of the procedure.

21. The method of claim 20, further comprising:
detecting, by one or more of the processors, a shape formed by the flexible elongate device within the environment; and wherein:
the graphical representation depicts the force encountered by the distal portion of the flexible elongate device in the detected shape formed by the flexible elongate device.
